(19) United States
(12) Patent Application Publication
(10) Pub. No.: US 2024/0158840 A1
(43) Pub. Date: May 16, 2024

(54) SYSTEMS AND METHODS FOR DETERMINING BARCODES AND SCREENING IN SITU

(71) Applicant: YALE UNIVERSITY, New Haven, CT (US)

(72) Inventors: Siyuan WANG, New Haven, CT (US); Bing YANG, New Haven, CT (US); Mengwei HU, New Haven, CT (US)

(73) Assignee: YALE UNIVERSITY, New Haven, CT (US)

(21) Appl. No.: 18/282,206

(22) PCT Filed: Mar. 16, 2022
(86) PCT No.: PCT/US22/20546

§ 371 Date: Sep. 14, 2023

Related U.S. Application Data
(60) Provisional application No. 63/162,257, filed on Mar. 17, 2021.

(51) Int. Cl. C12Q 1/6841; C12N 15/11; C12N 15/113; C12Q 1/6844; C12Q 1/6876

(52) U.S. Cl. CPC C12Q 1/6841; C12N 15/11; C12N 15/113; C12Q 1/6846; C12Q 1/6876; C12N 2310/20; C12N 2320/12

(57) ABSTRACT

Provided herein are methods for decoding nucleic acid barcodes in situ by using rolling circle amplification and labeled probes. Also provided herein are methods of performing an in situ genetic screen, using the nucleic acid barcode decoding techniques described herein.

### CROSS-REFERENCE TO RELATED APPLICATIONS

This patent application claims priority to U.S. Provisional Application No. 63/162,257, filed Mar. 17, 2021, the disclosure of each of which is herein incorporated by reference in its entirety.

### STATEMENT REGARDING FEDERALLY SPONSORED RESEARCH

This invention was made with government support under GM137414 awarded by National Institutes of Health. The government has certain rights in the invention.

### FIELD

Embodiments described herein generally relate to methods for amplifying and detecting nucleic acid barcodes in situ, and to methods for high-throughput in situ screening using the same strategy.

### SEQUENCE LISTING

The instant application contains a Sequence Listing which has been submitted electronically in ASCII format and is hereby incorporated by reference in its entirety. Said ASCII copy, created on Mar. 15, 2022, is named 251609_000062_SL.txt and is 751 bytes in size.

### BACKGROUND

Pooled genetic screens, such as CRISPR or RNAi screens, are powerful methods to discover genetic factors that affect various biomedical processes. Current pooled genetic screens are largely focused on growth or gene expression phenotypes. For example, a pool of cells with various genetic perturbations undergoes selection, either through the biomedical process or by cell sorting, and the remaining cells are sequenced to reveal the perturbations enriched/depleted through the selection. However, pooled genetic screen techniques that allow robust, high-throughput, and flexible screening of factors regulating in situ phenotypes are lacking. The in situ phenotypes include cell size, cell shape, cellular interactions, cellular abundance/density of biomolecules, sub-cellular abundance/density of biomolecules, sub-cellular morphologies of cellular compartments and organelles, sub-cellular distribution and organization of biomolecules. For example, genome architectures such as chromatin folding patterns are important in situ phenotypes. Correct three-dimensional organization of chromatin is essential for the proper functioning of cells in the human body. Defective chromatin organization can alter cellular behavior and is a hallmark of aging and multiple diseases including cancer and progeria, among others. Despite its critical importance, understanding how the three-dimensional organization of chromatin is regulated at the molecular level in health and disease remains a major challenge for the scientific and biomedical community. Thus, what is needed is a pooled genetic in situ screen that allows the screening of regulators of in situ phenotypes.

### SUMMARY

In some embodiments, provided is a method of decoding a nucleic acid barcode in situ in a sample, comprising the following steps:
a) amplifying at least a target region of a segment of the nucleic acid barcode by rolling circle amplification to generate amplified nucleic acids comprising copies of the segment target region, wherein the segment target region comprises one of a plurality of unique primary decoder sequences;
b) contacting the sample with one or more labeled readout probes under conditions that allow hybridization of said labeled readout probes to said amplified nucleic acids, wherein each said labeled readout probe comprises a sequence complementary to a sequence in said amplified nucleic acids;
c) detecting the label(s) of the one or more labeled readout probes; and
d) determining, based on the presence and/or identity of the labeled readout probe, the identity of the nucleic acid barcode.

In some embodiments of the method described above, the labeled readout probe comprises a sequence complementary to one of the plurality of unique primary decoder sequences.

In some embodiments of the methods described above, the nucleic acid barcode comprises a plurality of segments, and each segment comprises a target region comprising one of a unique plurality of unique primary decoder sequences, and wherein
step (a) comprises amplifying at least a target region of each segment of the nucleic acid barcode to generate a set of amplified nucleic acids,
step (b) comprises contacting the sample with one or more labeled readout probes under conditions that allow hybridization of said labeled readout probes to said amplified nucleic acids amplified from a first segment,
step (d) comprises determining, based on the presence and/or identity of the labeled readout probe from each segment, the identity of the nucleic acid barcode, and
wherein the method further comprises the following steps after step (c) and prior to step (d):
e) optionally eliminating signal from the label(s) of the readout probe detectable in step (c); and
f) repeating steps (b), (c), and (e) until the presence and/or identity of the labeled readout probe has been determined for each segment.

In some embodiments of the methods described above, step (a) comprises the following steps:
a1) contacting the sample with a pair of oligonucleotide probes under conditions that allow hybridization of said oligonucleotide probes to their respective target sequences, said pair of oligonucleotide probes comprising:
(i) a padlock probe comprising at least a region that is complementary to a first part of the segment of the nucleic acid barcode, wherein the padlock probe comprises a 5′ end region and 3′ end region and wherein upon said hybridization the 5′ and 3′ end regions of the padlock probe are brought into juxtaposition for circularization of the padlock probe, and wherein when circularized the padlock probe comprises the reverse complementary sequence of the segment target region; and
(ii) a linear probe comprising a region that is complementary to at least a region of the padlock probe;
a2) circularizing the padlock probe to form a circular padlock probe; and
a3) amplifying the circular padlock probe in situ to generate the amplified nucleic acids comprising copies of the segment target region.

In some embodiments of the methods described above, the linear probe comprises a binding region and an overhang region, wherein the binding region is complementary to a second part of the segment of the nucleic acid barcode and the overhang region is complementary to the at least a region of the padlock probe.

In some embodiments of the methods described above, when the nucleic acid barcode comprises a plurality of segments, the second part of each segment comprises a unique sequence. In some embodiments of the methods described above, when the nucleic acid barcode comprises a plurality of segments, the second part of each segment comprises the same sequence.

In some embodiments of the methods described above, the linear probe used for each segment comprises the same sequence. In some embodiments of the methods described above, the 5′ and 3′ end regions of the padlock probe are hybridized to the overhang region of the linear probe. In some embodiments of the methods described above, the 5′ and 3′ end regions of the padlock probe are hybridized to the first part of the segment of the nucleic acid barcode. In some embodiments of the methods described above, the linear probe does not comprise a binding region that is complementary to any part of the nucleic acid barcode. In some embodiments of the methods described above, the segment does not comprise a second part.

In some embodiments of the methods described above, upon padlock probe hybridization, the 5′ and 3′ end regions of the padlock probe are immediately next to each other, and wherein the circularizing step is performed by ligating the 5′ and 3′ end regions of the padlock probe with a ligase. In some embodiments of the methods described above, upon padlock probe hybridization, the 5′ and 3′ end regions of the padlock probe have a gap of one or more nucleotides between each other, and wherein the circularizing step is performed by gap filling with a polymerase or a reverse transcriptase and ligating the 5′ and 3′ end regions of the padlock probe with a ligase. In some embodiments of the methods described above, when the 5′ and 3′ end regions of the padlock probe are hybridized to the first part of the segment of the nucleic acid barcode, the length of the gap is shorter than the length of the target region. In some embodiments of the methods described above, when the 5′ and 3′ end regions of the padlock probe are hybridized to the first part of the segment of the nucleic acid barcode, the length of the gap is the same or longer than the length of the target region. In some embodiments of the methods described above, the padlock probe further comprises one of a plurality of unique secondary decoder sequences, wherein each said unique secondary decoder sequence is matched with one of the plurality of unique primary decoder sequences, and wherein the unique secondary decoder sequence is also amplified during rolling circle amplification.

In some embodiments of the methods described above, each said labeled readout probe comprises a sequence complementary to one of the plurality of unique secondary decoder sequences.

In some embodiments, provided is a method of decoding a nucleic acid barcode in situ in a sample, wherein each nucleic acid barcode comprises a plurality of segments, each segment comprising a target region comprises one of a unique plurality of unique primary decoder sequences, the method comprising the following steps:
a) contacting the sample with a plurality of pairs of oligonucleotide probes under conditions that allow hybridization of said pairs of oligonucleotide probes to their respective target sequences in each segment, each said pair of oligonucleotide probes comprising:
(i) a padlock probe comprising a 5′ end region, a 3′ end region, and a binding region that is complementary to a first part of the segment of the nucleic acid barcode, wherein said first part of the segment comprises a target region of a segment of the nucleic acid barcode and said segment target region comprises one of a plurality of unique primary decoder sequences; and
(ii) a linear probe comprising a binding region and an overhang region, wherein the binding region is complementary to the second part of the segment, wherein the 5′ end and 3′ end regions of the padlock probe are hybridized to the overhang region of the linear probe and wherein upon said hybridization the 5′ and 3′ end regions of the padlock probe are brought into juxtaposition for circularization of the padlock probe;
b) circularizing the padlock probes to form circular padlock probes;
c) amplifying the circular padlock probes in situ to generate a set of amplified nucleic acids comprising copies of the segment target region;
d) contacting the sample with one or more labeled readout probes under conditions that allow hybridization of said labeled readout probes to said amplified nucleic acids amplified from a first segment, wherein each said labeled readout probe comprises a sequence complementary to one of the plurality of the unique primary decoder sequences in the first segment target region;
e) detecting the label(s) of the one or more labeled readout probes;
f) optionally eliminating signal from the label(s) of the readout probe detectable in step (e);
g) repeating steps (d), (e), and (f) until the presence and/or identity of the labeled readout probe has been determined for each segment; and
h) determining, based on the presence and/or identity of the labeled readout probe from each segment, the identity of the nucleic acid barcode.

In some embodiments of the methods described above, the barcode comprises 1 to 100 segments. In some embodiments of the methods described above, the barcode comprises about 10 segments. In some embodiments of the methods described above, wherein the number of unique primary decoder sequences for each segment is about 2 to about 10000. In some embodiments of the methods described above, the number of unique primary decoder sequences for each segment is 3. In some embodiments of the methods described above, the length of each segment is about 15 nucleotides to about 10000 nucleotides. In some embodiments of the methods described above, the length of each segment is about 40 nucleotides. In some embodiments of the methods described above, each segment is separated by a spacer. In some embodiments of the methods described above, the length of the spacer is about 0 nucleotide to about 5000 nucleotides.

In some embodiments, provided is a method of decoding a nucleic acid barcode in situ in a sample, comprising the following steps:
a) amplifying at least a target region of a nucleic acid barcode by rolling circle amplification to generate amplified nucleic acids comprising copies of the target region, wherein the target region comprises a primary variable sequence;
b) contacting the sample with a plurality of encoding probes under conditions that allow hybridization of said encoding probes to said amplified nucleic acids, wherein at least one of said encoding probe comprises a sequence complementary to a sequence in said amplified nucleic acids, and each said encoding probe comprises one or more of a plurality of unique readout regions;
c) contacting the sample with one or more labeled readout probes under conditions that allow hybridization of said labeled readout probes to said encoding probe(s), wherein each said labeled readout probe comprises a sequence complementary to a unique readout region;
d) detecting the label(s) of the one or more labeled readout probes;
e) optionally eliminating signal from the label(s) of the readout probes detectable in step (d);
f) optionally repeating steps (c), (d) and (e) with one or more additional labeled readout probes, each comprising a sequence complementary to a different unique readout region; and
g) determining, based on the presence of the one or more labeled readout probes, the identity of the nucleic acid barcode.

In some embodiments of the methods described above, the nucleic acid barcode comprises only one target region. In some embodiments of the methods described above, at least one of said encoding probes comprises a sequence complementary to the primary variable sequence. In some embodiments of the methods described above, each encoding probe comprises two or more of a plurality of unique readout regions. In some embodiments of the methods described above, each encoding probe comprises one of a plurality of unique readout regions. In some embodiments of the methods described above, the nucleic acid barcode is from a library of nucleic acid barcodes.

In some embodiments of the methods described above, step (a) comprises the following steps:
a1) contacting the sample with a pair of oligonucleotide probes under conditions that allow hybridization of said oligonucleotide probes to their respective target sequences, said pair of oligonucleotide probes comprising:
(i) a padlock probe comprising at least a region that is complementary to a first part of the nucleic acid barcode, wherein the padlock probe comprises a 5′ end region and 3′ end region and wherein upon said hybridization the 5′ and 3′ end regions of the padlock probe are brought into juxtaposition for circularization of the padlock probe, and wherein when circularized the padlock probe comprises the reverse complementary sequence of the target region; and
(ii) a linear probe comprising a region that is complementary to at least a region of the padlock probe;
a2) circularizing the padlock probe to form a circular padlock probe; and
a3) amplifying the circular padlock probe in situ to generate the amplified nucleic acids comprising copies of the target region.

In some embodiments of the methods described above, the linear probe comprises a binding region and an overhang region, wherein the binding region is complementary to a second part of the nucleic acid barcode and the overhang region is complementary to at least a region of the padlock probe. In some embodiments of the methods described above, the 5′ and 3′ end regions of the padlock probe are hybridized to the overhang region of the linear probe. In some embodiments of the methods described above, the 5′ and 3′ end regions of the padlock probe are hybridized to the first part of the nucleic acid barcode. In some embodiments of the methods described above, the linear probe does not comprise a binding region that is complementary to any part of the nucleic acid barcode.

In some embodiments of the methods described above, when the nucleic acid barcode is from a library of nucleic acid barcodes, the second part of each nucleic acid barcode comprises a unique sequence. In some embodiments of the methods described above, when the nucleic acid barcode is from a library of nucleic acid barcodes, the second part of each nucleic acid barcode comprises the same sequence. In some embodiments of the methods described above, the nucleic acid barcode does not comprise a second part.

In some embodiments of the methods described above, upon padlock probe hybridization, the 5′ and 3′ end regions of the padlock probe are immediately next to each other, and wherein the circularizing step is performed by ligating the 5′ and 3′ end regions of the padlock probe with a ligase. In some embodiments of the methods described above, upon padlock probe hybridization, the 5′ and 3′ end regions of the padlock probe have a gap of one or more nucleotides between each other, and wherein the circularizing step is performed by gap filling with a polymerase or a reverse transcriptase and ligating the 5′ and 3′ end regions of the padlock probe with a ligase. In some embodiments of the methods described above, when the 5′ and 3′ end regions of the padlock probe are hybridized to the first part of the nucleic acid barcode, the length of the gap is shorter than the length of the target region. In some embodiments of the methods described above, when the 5′ and 3′ end regions of the padlock probe are hybridized to the first part of the nucleic acid barcode, the length of the gap is the same or longer than the length of the target region.

In some embodiments of the methods described above, the padlock probe further comprises a secondary variable sequence, wherein said secondary variable sequence is matched with the primary variable sequence, and wherein the secondary variable sequence is also amplified during rolling circle amplification. In some embodiments of the methods described above, at least one of said encoding probes comprises a sequence complementary to the secondary variable sequence.

In some embodiments, provided is a method of decoding a nucleic acid barcode in situ in a sample, wherein the nucleic acid barcode comprises only one target region, said target region comprises a primary variable sequence, said method comprising the following steps:
a) contacting the sample with a pair of oligonucleotide probes under conditions that allow hybridization of said oligonucleotide probes to their respective target sequences, said pair of oligonucleotide probes comprising:
(i) a padlock probe comprising a 5′ end region, a 3′ end region, and a binding region that is complementary to a first part of the nucleic acid barcode, wherein said first part comprises the target region of the nucleic acid barcode; and
(ii) a linear probe comprising a binding region and an overhang region, wherein the binding region is complementary to the second part of the nucleic acid barcode, wherein the 5′ end and 3′ end regions of the padlock probe are hybridized to the overhang region of the linear probe and wherein upon said hybridization the 5′ and 3′ end regions of the padlock probe are brought into juxtaposition for circularization of the padlock probe;
b) circularizing the padlock probes to form circular padlock probes;
c) amplifying the circular padlock probe in situ to generate amplified nucleic acids comprising copies of the target region;
d) contacting the sample with a plurality of encoding probes under conditions that allow hybridization of said encoding probes to said amplified nucleic acids, wherein at least one of said encoding probe comprises a sequence complementary to the primary variable sequence, and each said encoding probe comprises one or more of a plurality of unique readout regions;
e) contacting the sample with one or more labeled readout probes under conditions that allow hybridization of said labeled readout probes to said encoding probe(s), wherein each said labeled readout probe comprises a sequence complementary to a unique readout region;
f) detecting the label(s) of the one or more labeled readout probes;
g) optionally eliminating signal from the label(s) of the readout probe(s) detectable in step (f);
h) optionally repeating steps (e), (f) and (g) with one or more additional labeled readout probes, each comprising a sequence complementary to a different unique readout region; and
i) determining, based on the presence of the one or more labeled readout probes, the identity of the nucleic acid barcode.

In some embodiments of the methods described above, each encoding probe comprises two or more of a plurality of unique readout regions. In some embodiments of the methods described above, each encoding probe comprises one of a plurality of unique readout regions.

In some embodiments of the methods described above, the nucleic acid barcode is from a library of nucleic acid barcodes. In some embodiments of the methods described above, the second part of each nucleic acid barcode comprises a unique sequence. In some embodiments of the methods described above, the second part of each nucleic acid barcode comprises the same sequence.

In some embodiments of the methods described above, the number of unique readout regions is about 2 to 6000. In some embodiments of the methods described above, the length of the variable sequence is about 15 nucleotides to about 300 nucleotides. In some embodiments of the methods described above, the length of the variable sequence is about 20 nucleotides.

In some embodiments of the methods described above, the linear and/or padlock probes are single-stranded DNA. In some embodiments of the methods described above, the linear and/or padlock probes are single-stranded locked nucleic acid (LNA) or single-stranded DNA with partial LNA modification(s). In some embodiments of the methods described above, the linear and padlock probes are added simultaneously. In some embodiments of the methods described above, the linear and padlock probes are added sequentially.

In some embodiments of the methods described above, the amplification step is performed with a rolling circle amplification DNA polymerase. In some embodiments of the methods described above, the rolling circle amplification DNA polymerase is Phi29, Bst, or Vent exo-DNA polymerase. In some embodiments of the methods described above, the amplification step is performed with a rolling circle amplification RNA polymerase. In some embodiments of the methods described above, the rolling circle amplification RNA polymerase is T7 RNA polymerase.

In some embodiments of the methods described above, the circularization step comprises ligation with a ligase. In some embodiments of the methods described above, the ligase is a DNA ligase. In some embodiments of the methods described above, the DNA ligase is a T4 DNA ligase, T7 DNA ligase, T3 DNA ligase, Taq DNA ligase, Ampligase, or E. coli DNA ligase. In some embodiments of the methods described above, the ligase is a SplintR ligase.

In some embodiments of the methods described above, the circularization of the padlock probe is performed in situ. In some embodiments of the methods described above, the circularization of the padlock probe is performed in vitro prior to contacting the sample with the padlock probe.

In some embodiments of the methods described above, the readout probes are labeled with fluorescent dyes. In some embodiments of the methods described above, at least some readout probes are labeled with the same fluorescent dye. In some embodiments of the methods described above, at least some readout probes are labeled with different fluorescent dyes.

In some embodiments of the methods described above, the fluorescence signal is eliminated by photobleaching, chemical bleaching, chemical cleavage, chemical wash, heat denaturation, nuclease treatment, or a combination thereof. In some embodiments of the methods described above, the fluorescence signal is retained.

In some embodiments of the methods described above, the amplified nucleic acids are crosslinked to the sample. In some embodiments of the methods described above, the crosslinking is performed by aminoallyl-dUTP spike-in during the amplification step, and post-fixation with paraformaldehyde and/or PEGylated bis(sulfosuccinimidyl)suberate (BS(PEG)5 or BS(PEG)9).

In some embodiments of the methods described above, the nucleic acid barcode is comprised in a DNA, RNA, locked nucleic acid (LNA), DNA with partial LNA modification(s), or peptide nucleic acid (PNA) molecule. In some embodiments of the methods described above, the nucleic acid molecule is double-stranded. In some embodiments of the methods described above, the nucleic acid molecule is single-stranded.

In some embodiments of the methods described above, the nucleic acid barcode is delivered into cells. In some embodiments of the methods described above, the barcode is not delivered into cells but is decoded on the surface of cells or independent of cells. In some embodiments of the methods described above, the barcode is decoded at a molecular level, cellular level, or multi-cellular level.

In some embodiments, provided is a method of performing an in situ genetic screen, comprising the following steps:
pairing a genetic screen technique with nucleic acid barcodes;
performing the genetic screen technique; and
decoding the nucleic acid barcodes with any decoding method described herein.

In some embodiments of the methods described above, the genetic screen technique is a pooled genetic screen technique. In some embodiments of the methods described above, the genetic screen technique is a CRISPR screen technique. In some embodiments of the methods described above, the CRISPR screen is a CRISPR knockout screen, a CRISPR interference (CRISPRi) screen, a CRISPR activation (CRISPRa) screen, a CRISPR screen of cis-regulatory elements, a CRISPR screen of protein domain functions, or a CRISPR double-perturbation screen. In some embodiments of the methods described above, the genetic screen technique is an RNA interference (RNAi) screen technique. In some embodiments of the methods described above, the genetic screen technique is a massively parallel reporter assay screen.

In some embodiments of the methods described above, the step of pairing a genetic screen technique with nucleic acid barcodes further comprises pairing at least one nucleic acid barcode with at least one nucleic acid genetic perturbation sequence. In some embodiments of the methods described above, each barcode pairs with a unique genetic perturbation sequence. In some embodiments of the methods described above, each barcode and genetic perturbation sequence pairing are located on one polynucleotide sequence. In some embodiments of the methods described above, the nucleic acid barcode is attached to the genetic perturbation sequence. In the embodiments of the methods described above, the nucleic acid barcode is the genetic perturbation sequence. In some embodiments of the methods described above, each barcode and genetic perturbation sequence pairing are located on multiple polynucleotide sequences.

In some embodiments of the methods described above, the step of pairing a genetic screen technique with nucleic acid barcodes comprises pairing at least one nucleic acid barcode with a combination of at least two nucleic acid genetic perturbation sequences. In some embodiments of the methods described above, the genetic perturbation sequence is a guide RNA (gRNA). In some embodiments of the methods described above, each genetic perturbation sequence is a unique gRNA.

In some embodiments of the methods described above, the nucleic acid barcodes and nucleic acid genetic perturbation sequences are delivered into cells. In some embodiments of the methods described above, the nucleic acid barcodes and nucleic acid genetic perturbation sequences are delivered into cells by viral transduction, transfection, electroporation, or microinjection. In some embodiments of the methods described above, the nucleic acid barcodes and nucleic acid genetic perturbation sequences are delivered into cells by viral transduction. In some embodiments of the methods described above, viral transduction is performed by a lentivirus or an adeno-associated virus (AAV).

In some embodiments of the methods described above, the method further comprises analyzing the results of the genetic screen technique to determine a phenotypic perturbation. In some embodiments of the methods described above, the phenotypic perturbation is a perturbation of cell size, cell shape, cellular interactions, cellular abundance/density of biomolecules, sub-cellular abundance/density of biomolecules, sub-cellular morphologies, sub-cellular distribution, and/or sub-cellular organization. In some embodiments of the methods described above, the phenotypic perturbation is a perturbation of genome architecture. In some embodiments of the methods described above, the phenotypic perturbation is a perturbation of three-dimensional chromatin organization.

In some embodiments of the methods described above, the analysis of the results of the genetic screen technique is performed by an imaging technique. In some embodiments of the methods described above, the imaging technique is in situ hybridization. In some embodiments of the methods described above, the imaging technique is fluorescence in situ hybridization. In some embodiments of the methods described above, the imaging technique is multiplexed DNA or RNA fluorescence in situ hybridization. In some embodiments of the methods described above, the imaging technique is imaging of lipid, sugar, metabolite, DNA, RNA, protein and/or DNA/RNA/protein modifications.

In some embodiments of the methods described above, the method further comprises the step of matching the decoded nucleic acid barcodes with the determined phenotypic perturbation. In some embodiments of the methods described above, the matching of the decoded barcode with the phenotypic perturbation allows for the determination of which genetic perturbation sequence matches which phenotypic perturbation. In some embodiments of the methods described above, the step of analyzing the results of the genetic screen technique to determine a phenotypic perturbation can be performed prior to, during, or after the decoding step.

In some embodiments, provided is a method of performing an in situ genetic screen, comprising the following steps:
creating at least one unique pairing of at least one nucleic acid barcode with at least one nucleic acid genetic perturbation sequence;
introducing at least one unique pairing of the at least one barcode and the at least one perturbation sequence pairing to a cell;
incubating the cell under conditions that allow the at least one perturbation sequence to cause the cell to display at least one phenotypic perturbation;
analyzing the cell by an imaging technique to determine the at least one phenotypic perturbation;
decoding the at least one nucleic acid barcode with any decoding method described herein; and
determining the at least one genetic perturbation sequence that causes the cell to display the at least one phenotypic perturbation.

In some embodiments, provided is a method of determining cellular positions in a single-cell sequencing, comprising the following steps:
introducing at least one nucleic acid barcode to at least one cell;
imaging the at least one cell to determine cellular position;
decoding the nucleic acid barcodes with any decoding method described herein;
dissociating the at least one cell from its substrate;
performing single-cell sequencing on the at least one cell to determine at least the sequence of the nucleic acid barcode associated with the at least one cell; and
mapping the at least one cell to the cellular position.

In some embodiments of the method described above, the at least one nucleic acid barcode is comprised in a DNA, RNA, locked nucleic acid (LNA), DNA with partial LNA modification(s) or peptide nucleic acid (PNA) molecule. In some embodiments of the methods described above, the at least one nucleic acid barcode is delivered by a viral vector. In some embodiments of the methods described above, the viral vector is a lentivirus or adeno-associated virus (AAV).

In some embodiments of the methods described above, the method comprises introducing a plurality of nucleic acid barcodes to a plurality of cells, wherein each nucleic acid barcode is only present in one cell. In some embodiments of the methods described above, each nucleic acid barcode is a unique nucleic acid barcode. In some embodiments of the methods described above, the at least one cell is present in at least one tissue.

In some embodiments of the methods described above, the step of performing single-cell sequencing on the at least one cell further determines additional genomic information about the at least one cell. In some embodiments of the methods described above, the step of performing single-cell sequencing on the at least one cell further determines the gene expression of the at least one cell. In some embodiments of the methods described above, the step of performing single-cell sequencing on the at least one cell further determines epigenetic/epigenomic information about the at least one cell. In some embodiments of the methods described above, the step of mapping the at least one cell to the cellular position provides spatial-omic information about the at least one cell.

### BRIEF DESCRIPTION OF THE FIGURES

FIG. 1 depicts a concept of an image-based pooled screen of chromatin organization regulators.

FIGS. 2A-2D depict an experimental design of an image-based pooled screen of chromatin-organization regulators. FIG. 2A depicts the plasmid library construction, with each gRNA paired with a unique barcode. FIG. 2B depicts cells after lentiviral transduction, Cas9-mediated disruption, and selection. FIG. 2C depicts multiplexed sequential DNA FISH to identify chromatin phenotypes. FIG. 2D depicts multiplexed sequential RNA FISH to identify the barcodes.

FIG. 3 depicts a BARC-FISH amplification design based on a 10-trit barcode. The barcode contains ten 40-nt DNA fragments, separated by a single nucleotide. Each 40-base fragment contains two regions: after being transcribed into mRNA, one half of the fragment is partially complementary to a linear probe, and the other half is partially complementary to a padlock probe. The linear probe and padlock probe are also partially complementary to each other. By in situ ligation and elongation (through rolling circle amplification), the region of barcode to which the padlock probe is hybridized will be amplified, which can be further visualized by applying dye-labeled readout probes. Each digit is assigned with three different sequences, referred to as three values, which will be visualized in different fluorescence channels and/or hybridization rounds.

FIG. 4 depicts the use of BARC-FISH to detect test barcode #1. The 9 digits of one field of view are shown. Four EGFP+ cells were analyzed in this field. In digit-1 (the first row-first round of imaging) BARC-FISH signal only appeared in the value-1 channel, as highlighted by a rectangle. The readout probes for digit-1 are then removed and readout probes for digit-2 are introduced and hybridized to the cells. In digit-2 (the second row-second round of imaging) BARC-FISH signal appeared mainly in the value-2 channel, as highlighted by a rectangle. All 9 rounds of hybridization and imaging are performed to read out the identity of 9 digits. Altogether, the cells are decoded as 121212121, which is consistent with the identity of test barcode #1.

FIG. 5 depicts the use of BARC-FISH to distinguish mixed cell populations carrying different barcodes. All 10 digits of one representative field of view in the decoding dataset used in FIG. 6 are shown. EGFP+ cells (imaged in 488 channel, first panel in the first row) express test barcode #1. mCherry+ cells (imaged in 560 channel, first panel in the second row) express test barcode #2. For each digit, the top row shows the value 1 signal, and the bottom row shows the value 2 signal. Note that the dye-labeled readout probe for the value 2 of digit 10 was not available when the experiment was conducted. EGFP+ cells can be successfully decoded as 1212121212, and mCherry+ cells can be successfully decoded as 2121212121, as designed. Scale bar: 40 μm.

FIG. 6 depicts bar graphs showing the correct decoding rate of BARC-FISH. EGFP+ cells expressing test barcode #1 on the top panel, mCherry+ cells expressing test barcode #2 on the bottom panel.

FIG. 7 depicts a test of linear and padlock probes comprising single-stranded DNA with partial LNA modifications in BARC-FISH. One representative field of view of a CRISPR screen library transduced cell culture is shown. 488 picture (upper left) represents cell auto-fluorescence. Value 1 (upper right) and value 2 (lower right) pictures are images of digit 10. Two distinct cell populations carrying different values can be distinguished.

FIGS. 8A-8C depict an overview of a BARC-FISH 2 design. BARC-FISH 2 uses a single segment with numerous varieties, combinatorially encodes the numerous varieties in an encoding hybridization, and decodes the numerous varieties with multiplexed FISH. FIG. 8A depicts a design where each encoding probe carries the full combinatorial code. FIG. 8B depicts a design where each encoding probe carries part of the combinatorial code. FIG. 8C depicts a demonstration of reading out 2 digits from the same rolling circle amplification (RCA) products.

FIGS. 9A-9C depict alternative padlock probe circularization and ligation strategies. FIG. 9A depicts an example where the ends of the padlock probe can be directly hybridized to and ligated upon the barcode segment. FIG. 9B depicts an example where after hybridization of the padlock probe, its ends could be one or multiple nucleotides away from each other, leaving a gap in between, which can be filled before the ligation reaction. The gap could span part of or the entire barcode segment. FIG. 9C depicts an example where the readout/encoding probe may target an alternative variable region (thick black bars) on the RCA product that is amplified from a variable region on the padlock probe (thick light gray bar aligned with thick black bars) with different sequences corresponding to the barcode sequences.

FIG. 10 depicts a design of a CRISPR screen with double perturbations with BARC-FISH. For simplicity, only the plasmid library design and three plasmids are shown.

FIG. 11 depicts a design of using BARC-FISH to mark cellular positions in single-cell sequencing. Here, BARC-FISH (of any type) is used to image cells in a tissue, before the cells are dissociated for sequencing. The sequencing data is then mapped back onto the tissue image.

FIG. 12 depicts an example of BARC-FISH 1 decoding in a CRISPR screen. Top left panel: the processed image of one representative field of view (FOV) showing value 0 of digit 1 of the BARC-FISH 1 experiment. The black dashed box in the panel indicates a representative cell. The processed images of this cell in all 3 values (3 color channels) across all 10 digits (10 rounds of sequential FISH) are shown in the other panels. BARC-FISH foci (white dots), cell body (grayscale) and cell segmentation (white lines) are overlaid in each panel. The boxed cell is decoded as: 0100122110, corresponding to one sgRNA targeting gene IKBKG in the screen library. Scale bars: 40 μm (whole FOV) and 10 μm (zoom in).

FIG. 13 depicts BARC-FISH 1 decoding combined with multi-modal imaging of molecular and cellular features in a CRISPR screen. The top row shows four imaging features from one FOV. White boxes indicate the same cell. The three zoomed-in views on the bottom row show labeling of topologically associating domains (TADs) 1, 3 and 16 respectively during the chromatin tracing procedure (multiplexed sequential DNA FISH), which sequentially visualizes all 27 TADs along human chromosome 22 (chr22). The folding conformation of one copy of chr22 in the cell indicated by the white arrows is displayed in the last panel. Scale bar: 40 μm (whole FOV) and 5 μm (zoom-in).

FIG. 14 depicts a volcano plot of adjacent TAD distance change upon gene knock-out in the BARC-FISH 1 screen. Each dot represents one sgRNA (one gene perturbation). The intensities of the dots represent the numbers of chromatin traces detected in the screen. For each perturbation, the distance between each pair of adjacent TADs on chr22 was calculated for each chromatin trace. Paired t-tests with correction for multiple hypothesis testing were performed on the mean values of the adjacent TAD distances between each sgRNA and non-targeting control sgRNAs to compute the false discovery rate of each sgRNA. The log 2 fold change of the mean distance between each sgRNA and non-targeting control sgRNAs was calculated by taking the mean value of the fold changes of adjacent TAD distances between the sgRNA and control sgRNAs and performing log transformation. The vertical and horizontal dashed lines represent significance and fold change cutoffs for candidate selection, respectively.

FIG. 15 depicts an example of BARC-FISH 2 decoding in a CRISPR screen. Top left panel: fluorescent image of digit 1 from a representative FOV of a BARC-FISH 2 experiment. The black dashed box in the panel indicates a representative cell, and all 14 rounds of sequential decoding FISH images of the cell were shown in the other panels. This cell can be decoded as: 10011000000010, corresponding to one sgRNA targeting gene FBXL18 in the screen library. Scale bar: 40 μm (whole FOV) and 10 μm (zoom in).

FIG. 16 depicts BARC-FISH 2 decoding combined with multi-modal imaging of molecular and cellular features in a CRISPR screen. The top row shows four imaging features from one FOV. White boxes indicate the same cell. The three zoomed-in views on the bottom row show labeling of TAD1, 9 and 19 respectively during the chromatin tracing procedure, which sequentially visualizes all 27 TADs along chr22. The folding conformation of one copy of chr22 in the boxed cell indicated by the white arrows is displayed in the last panel. Scale bar: 40 μm (whole FOV) and 5 μm (zoom-in).

FIG. 17 depicts a volcano plot of radius of gyration change upon gene knock-out in BARC-FISH 2 screen. Each dot represents one sgRNA. The intensities of the dots show the numbers of chromatin traces in the whole screen. For each perturbation, the radius of gyration of chr22 was calculated for each chromatin trace that has at least 25 detected TADs. T-tests with correction for multiple hypothesis testing were performed on the radii of gyration between each sgRNA and non-targeting control sgRNAs to compute the false discovery rate of each sgRNA. The log 2 fold change of radius of gyration for each sgRNA was calculated by dividing the mean radius of gyration of each sgRNA by the mean radius of gyration of the control sgRNAs and performing log transformation. Dashed lines represent cutoffs for candidate selection.

### DETAILED DESCRIPTION

Provided here are methods for decoding nucleic acid barcodes in situ. Specifically, provided herein are methods for decoding nucleic acid barcodes in situ by amplifying a nucleic acid barcode by rolling circle amplification, and then contacting the amplified barcodes with labeled readout probes. Also provided herein are methods of performing an in situ genetic screen, wherein the method comprises amplifying a nucleic acid barcode by rolling circle amplification.

### Definitions

Unless defined otherwise, technical and scientific terms used herein have the same meaning as commonly understood by one of ordinary skill in the art to which this invention belongs. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

As used in this specification and the appended claims, the singular forms “a,” “an,” and “the” include plural referents unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of up to ±10% from the specified value, as such variations are appropriate to perform the disclosed methods. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

“Amplification,” as used herein, refers to any in vivo, in vitro, or in situ process for increasing the number of copies of a nucleotide sequence or sequences. As used herein, one amplification reaction may consist of many rounds of replication. For example, one rolling circle amplification reaction may consist of the creation of multiple copies of a target template or a section of the target template.

“Polynucleotide,” synonymously referred to as “nucleic acid molecule,” “nucleotides” or “nucleic acids,” refers to any polyribonucleotide or polydeoxyribonucleotide, which may be unmodified RNA or DNA or modified RNA or DNA. “Polynucleotide” also embraces relatively short nucleic acid chains, often referred to as “oligonucleotides.” Polynucleotides and oligonucleotides herein include, without limitation unless otherwise indicated, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The terms “polynucleotide” and “oligonucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, a “polynucleotide” embraces chemically, enzymatically or metabolically modified forms of polynucleotides and oligonucleotides as typically found in nature, as well as the chemical forms of DNA and RNA characteristic of viruses and cells.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. If aspects or embodiments of the disclosure are described as “comprising”, or versions thereof (e.g., comprises), a feature, embodiments also are contemplated “consisting of” or “consisting essentially of” the feature.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms and expressions which have been employed herein are used as terms of description and not of limitation, and use of such terms and expressions do not exclude any equivalents of the features shown and described or portions thereof, and various modifications are possible within the scope of the technology claimed.

The practice of the present disclosure employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ. Additional techniques are explained, e.g., in U.S. Pat. No. 7,912,698 and U.S. Patent Appl. Pub. Nos. 2011/0202322 and 2011/0307437.

### Methods

Methods for decoding a nucleic acid barcode in situ are provided herein. In general, nucleic acid barcodes are unique or semi-unique polynucleotides that comprise one or more segments, with each segment comprising one or more target sequences from a pool of potential sequences. When paired with another molecular construct or when inserted into a cell, the nucleic acid barcode allows the unique identification of the construct or cell when the barcode is identified using molecular techniques. For example, when nucleic acid barcodes are paired with DNA or RNA modification techniques such as CRISPR or RNAi for pooled genetic screens, identification of the barcodes will identify the specific genetic effect caused by the given modification technique.

In some embodiments, the method comprises a step of amplifying at least a target region of a segment of the nucleic acid barcode by rolling circle amplification. Rolling circle amplification is also known to those skilled in the art, and is a form of rolling circle replication used for the amplification of nucleic acids from some amount of starting material. In rolling circle amplification, a polymerase continuously adds nucleotides to a primer bound to a circular template, resulting in a ssDNA product that repeats all of some of the circular template.

In some embodiments, after rolling circle amplification of the nucleic acid barcodes is performed to create amplified nucleic acids, one or more labeled readout probes which comprise a sequence complementary to a sequence in the amplified nucleic acid are added. Once bound to the complementary amplified nucleic acids, the labeled readout probes are detected. Based on the presence and/or identity of each labeled readout probe, the identity of the nucleic barcode can be determined.

In some embodiments, the detection of the labeled readout probes is performed by an imaging method. In some embodiments, the method is fluorescence in situ hybridization (FISH). In some embodiments, any method described herein for determining nucleic acid barcodes can be entitled Barcode Amplification by Rolling Circle and readout by FISH (BARC-FISH).

In some embodiments, a method of decoding a nucleic acid barcode in situ in a sample is provided, the method comprising the steps of: a) amplifying at least a target region of a segment of the nucleic acid barcode by rolling circle amplification to generate amplified nucleic acids comprising copies of the segment target region, wherein the segment target region comprises one of a plurality of unique primary decoder sequences; b) contacting the sample with one or more labeled readout probes under conditions that allow hybridization of said labeled readout probes to said amplified nucleic acids, wherein each said labeled readout probe comprises a sequence complementary to a sequence in said amplified nucleic acids; c) detecting the label(s) of the one or more labeled readout probes; and d) determining, based on the presence and/or identity of the labeled readout probe, the identity of the nucleic acid barcode. In some embodiments, the labeled readout probe comprises a sequence complementary to one of the plurality of unique primary decoder sequences. For example, at least one labeled readout probe will be required for each unique primary decoder sequence used in the nucleic barcodes.

In some embodiments, the nucleic acid barcode comprises a plurality of segments, and each segment comprises a target region comprising one of a unique plurality of unique primary decoder sequences, and wherein step (a) described above comprises amplifying at least a target region of each segment of the nucleic acid barcode to generate a set of amplified nucleic acids, step (b) described above comprises contacting the sample with one or more labeled readout probes under conditions that allow hybridization of said labeled readout probes to said amplified nucleic acids amplified from a first segment, step (d) described above comprises determining, based on the presence and/or identity of the labeled readout probe from each segment, the identity of the nucleic acid barcode. In some embodiments, after step (c) and prior to step (d) above, the method further comprises the step of (e) optionally eliminating signal from the label(s) of the readout probe detectable in step (c); and (f) repeating steps (b), (c), and (e) until the presence and/or identity of the labeled readout probe has been determined for each segment. For example, it can be necessary to eliminate the signal from labels already detected to allow accurate detection of additional labels. This can be done, for example, by photo bleaching, chemical bleaching, chemical cleavage, chemical wash, heat denaturation, nuclease treatment or other imaging techniques to remove the signal strength from certain labels.

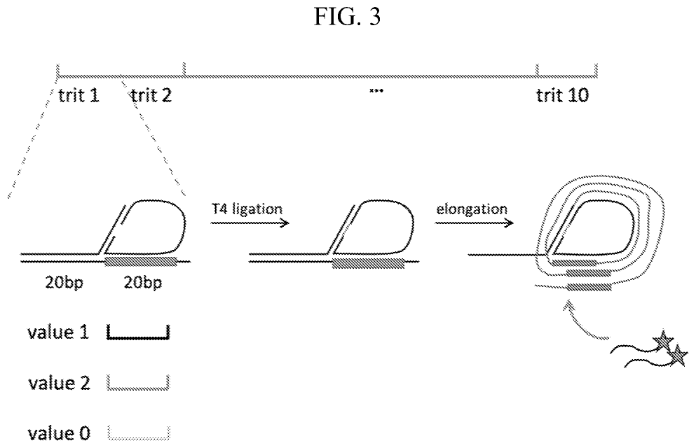

In some embodiments, the methods herein use at least one linear probe and at least one padlock probe to identify and amplify the target sequences by rolling circle amplification. In some embodiments, the linear probe is capable of binding to a portion of a nucleic acid barcode sequence and a corresponding padlock probe. In turn, the padlock probe is capable of binding to a different portion of the same nucleic acid barcode sequence and at least one section of the corresponding linear probe. The padlock probe is then circularized and rolling circle amplification can begin. An example of liner and padlock probe binding to a segment of a nucleic acid barcode is shown in FIG. 3. In general, padlock probes are a type of single-stranded molecular inversion probe that comprises two ends that complementary to different sections of a target sequence. When the padlock probe is hybridized to its target sequence, the padlock probe forms a completely circular or partially circular shape, depending on the presence of a gap between the ends of the probe. The use of the linear and padlock probe combination allows high target specificity, with a low chance of off-target amplification. Thus, in combination with the rolling circle amplification, the methods described herein deliver both a stronger and more specific signal over other barcode readout techniques known in the art. See, for example, the readout techniques as provided in U.S. Patent Publication Nos. 2017/02207331 and 2020/0095630, both of which are incorporated herein by reference in their entirety.

In some embodiments, any step (a) described above comprises the steps of a1) contacting the sample with a pair of oligonucleotide probes under conditions that allow hybridization of said oligonucleotide probes to their respective target sequences, said pair of oligonucleotide probes comprising: (i) a padlock probe comprising at least a region that is complementary to a first part of the segment of the nucleic acid barcode, wherein the padlock probe comprises a 5′ end region and 3′ end region and wherein upon said hybridization the 5′ and 3′ end regions of the padlock probe are brought into juxtaposition for circularization of the padlock probe, and wherein when circularized the padlock probe comprises the reverse complementary sequence of the segment target region; and (ii) a linear probe comprising a region that is complementary to at least a region of the padlock probe; a2) circularizing the padlock probe to form a circular padlock probe; and a3) amplifying the circular padlock probe in situ to generate the amplified nucleic acids comprising copies of the segment target region.

In some embodiments, the linear probe comprises a binding region and an overhang region, wherein the binding region is complementary to a second part of the segment of the nucleic acid barcode and the overhang region is complementary to the at least a region of the padlock probe. In some embodiments, the linear probe used for each segment comprises the same sequence. In some embodiments, when the nucleic acid barcode comprises a plurality of segments, the second part of each segment comprises a unique sequence. In some embodiments, when the nucleic acid barcode comprises a plurality of segments, the second part of each segment comprises the same sequence. In some embodiments, the linear probe used for each segment comprises the same sequence. For example, one potential design of nucleic acid barcodes includes, as a part of each segment of the barcode, a sequence that the linear probe can bind to. This design allows the use of just one type of linear probe per barcode sequence. A sequence that the linear probe can bind to can be present anywhere in each segment of the barcode, for example at the 5′ end of the segment or the 3′ end of the segment. In some embodiments, the linear probe does not comprise a binding region that is complementary to any part of the nucleic acid barcode. In some embodiments, the segment does not comprise a second part.

In some embodiments, the 5′ and 3′ end regions of the padlock probe are hybridized to the overhang region of the linear probe. In some embodiments, the 5′ and 3′ end regions of the padlock probe are hybridized to the first part of the segment of the nucleic acid barcode. In some embodiments, upon padlock probe hybridization, the 5′ and 3′ end regions of the padlock probe are immediately next to each other, and wherein the circularizing step is performed by ligating the 5′ and 3′ end regions of the padlock probe with a ligase. In some embodiments, upon padlock probe hybridization, the 5′ and 3′ end regions of the padlock probe have a gap of one or more nucleotides between each other, and wherein the circularizing step is performed by gap filling with a polymerase or a reverse transcriptase and ligating the 5′ and 3′ end regions of the padlock probe with a ligase. In some embodiments, the 5′ and 3′ end regions of the padlock probe are hybridized to the first part of the segment of the nucleic acid barcode, the length of the gap is shorter than the length of the target region. In some embodiments, the 5′ and 3′ end regions of the padlock probe are hybridized to the first part of the segment of the nucleic acid barcode, the length of the gap is the same or longer than the length of the target region.

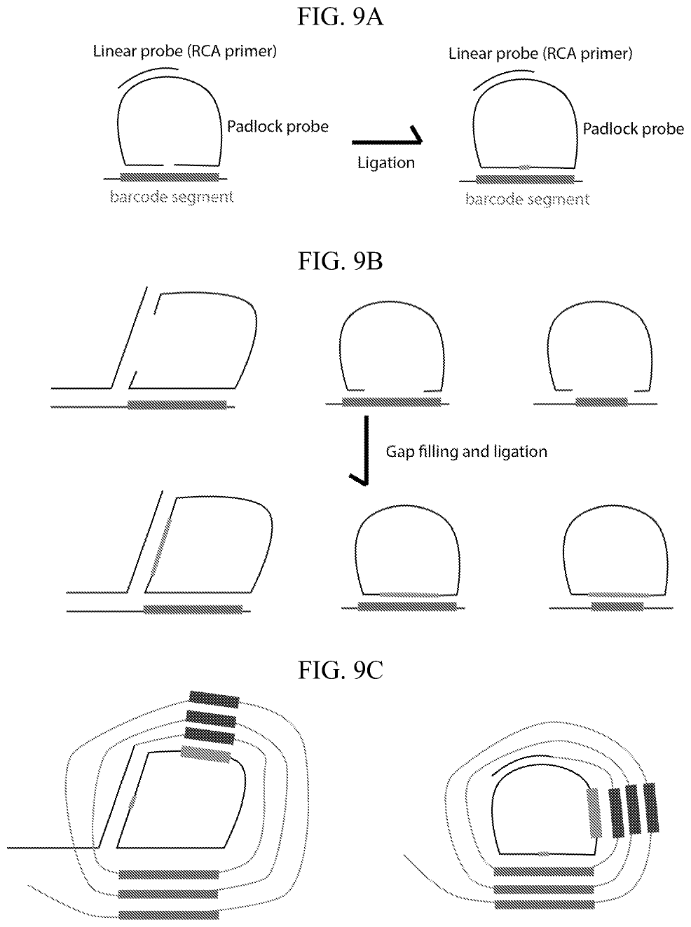

In some embodiments, the padlock probe further comprises one of a plurality of unique secondary decoder sequences, wherein each said unique secondary decoder sequence is matched with one of the plurality of unique primary decoder sequences, and wherein the unique secondary decoder sequence is also amplified during rolling circle amplification. As shown in FIG. 9C, it is possible for the padlock probe to comprise additional decoder sequences. Once the circularization of the padlock probe is complete, any additional decoder sequences would also be amplified by rolling circle amplification. In some embodiments, a labeled readout probe comprises a sequence complementary to one of the plurality of unique secondary decoder sequences.

In some embodiments, a method of decoding a nucleic acid barcode in situ in a sample is provided herein, wherein each nucleic acid barcode comprises a plurality of segments, each segment comprising a target region comprises one of a unique plurality of unique primary decoder sequences, the method comprising the steps of: a) contacting the sample with a plurality of pairs of oligonucleotide probes under conditions that allow hybridization of said pairs of oligonucleotide probes to their respective target sequences in each segment, each said pair of oligonucleotide probes comprising: (i) a padlock probe comprising a 5′ end region, a 3′ end region, and a binding region that is complementary to a first part of the segment of the nucleic acid barcode, wherein said first part of the segment comprises a target region of a segment of the nucleic acid barcode and said segment target region comprises one of a plurality of unique primary decoder sequences; and (ii) a linear probe comprising a binding region and an overhang region, wherein the binding region is complementary to the second part of the segment, wherein the 5′ end and 3′ end regions of the padlock probe are hybridized to the overhang region of the linear probe and wherein upon said hybridization the 5′ and 3′ end regions of the padlock probe are brought into juxtaposition for circularization of the padlock probe; b) circularizing the padlock probes to form circular padlock probes; c) amplifying the circular padlock probes in situ to generate a set of amplified nucleic acids comprising copies of the segment target region; d) contacting the sample with one or more labeled readout probes under conditions that allow hybridization of said labeled readout probes to said amplified nucleic acids amplified from a first segment, wherein each said labeled readout probe comprises a sequence complementary to one of the plurality of the unique primary decoder sequences in the first segment target region; e) detecting the label(s) of the one or more labeled readout probes; f) optionally eliminating signal from the label(s) of the readout probe detectable in step (e); g) repeating steps (d), (e), and (f) until the presence and/or identity of the labeled readout probe has been determined for each segment; and h) determining, based on the presence and/or identity of the labeled readout probe from each segment, the identity of the nucleic acid barcode.

In some embodiments, the barcode comprises about 1 to about 100 segments. In some embodiments, the barcode comprises about 5 to about 100 segments. In some embodiments, the barcode comprises about 10 to about 100 segments. In some embodiments, the barcode comprises about 15 to about 100 segments. In some embodiments, the barcode comprises about 20 to about 100 segments. In some embodiments, the barcode comprises about 25 to about 100 segments. In some embodiments, the barcode comprises about 30 to about 100 segments. In some embodiments, the barcode comprises about 35 to about 100 segments. In some embodiments, the barcode comprises about 40 to about 100 segments. In some embodiments, the barcode comprises about 45 to about 100 segments. In some embodiments, the barcode comprises about 50 to about 100 segments. In some embodiments, the barcode comprises about 55 to about 100 segments. In some embodiments, the barcode comprises about 60 to about 100 segments. In some embodiments, the barcode comprises about 65 to about 100 segments. In some embodiments, the barcode comprises about 70 to about 100 segments. In some embodiments, the barcode comprises about 75 to about 100 segments. In some embodiments, the barcode comprises about 80 to about 100 segments. In some embodiments, the barcode comprises about 85 to about 100 segments. In some embodiments, the barcode comprises about 90 to about 100 segments. In some embodiments, the barcode comprises about 95 to about 100 segments. In some embodiments, the barcode comprises about 1 to about 95 segments. In some embodiments, the barcode comprises about 1 to about 90 segments. In some embodiments, the barcode comprises about 1 to about 85 segments. In some embodiments, the barcode comprises about 1 to about 80 segments. In some embodiments, the barcode comprises about 1 to about 75 segments. In some embodiments, the barcode comprises about 1 to about 70 segments. In some embodiments, the barcode comprises about 1 to about 65 segments. In some embodiments, the barcode comprises about 1 to about 60 segments. In some embodiments, the barcode comprises about 1 to about 55 segments. In some embodiments, the barcode comprises about 1 to about 50 segments. In some embodiments, the barcode comprises about 1 to about 45 segments. In some embodiments, the barcode comprises about 1 to about 40 segments. In some embodiments, the barcode comprises about 1 to about 35 segments. In some embodiments, the barcode comprises about 1 to about 30 segments. In some embodiments, the barcode comprises about 1 to about 25 segments. In some embodiments, the barcode comprises about 1 to about 20 segments. In some embodiments, the barcode comprises about 1 to about 15 segments. In some embodiments, the barcode comprises about 1 to about 10 segments. In some embodiments, the barcode comprises about 1 to about 5 segments. In some embodiments, the barcode comprises 10 segments.

In some embodiments, the number of unique primary decoder sequences for each segment is about 2 to about 10000. In some embodiments, the number of unique primary decoder sequences for each segment is about 3 to about 10000. In some embodiments, the number of unique primary decoder sequences for each segment is about 4 to about 10000. In some embodiments, the number of unique primary decoder sequences for each segment is about 5 to about 10000. In some embodiments, the number of unique primary decoder sequences for each segment is about 10 to about 10000. In some embodiments, the number of unique primary decoder sequences for each segment is about 50 to about 10000. In some embodiments, the number of unique primary decoder sequences for each segment is about 100 to about 10000. In some embodiments, the number of unique primary decoder sequences for each segment is about 500 to about 10000. In some embodiments, the number of unique primary decoder sequences for each segment is about 1000 to about 10000. In some embodiments, the number of unique primary decoder sequences for each segment is about 5000 to about 10000. In some embodiments, the number of unique primary decoder sequences for each segment is about 2 to about 5000. In some embodiments, the number of unique primary decoder sequences for each segment is about 2 to about 1000. In some embodiments, the number of unique primary decoder sequences for each segment is about 2 to about 500. In some embodiments, the number of unique primary decoder sequences for each segment is about 2 to about 100. In some embodiments, the number of unique primary decoder sequences for each segment is about 2 to about 50. In some embodiments, the number of unique primary decoder sequences for each segment is about 2 to about 10. In some embodiments, the number of unique primary decoder sequences for each segment is about 2 to about 5. In some embodiments, the number of unique primary decoder sequences for each segment is about 2 to about 4. In some embodiments, the number of unique primary decoder sequences for each segment is about 2 to about 3. In some embodiments, the number of unique primary decoder sequences for each segment is 3.

In some embodiments, the length of each segment is about 15 nucleotides to about 10000 nucleotides. In some embodiments, the length of each segment is about 20 nucleotides to about 10000 nucleotides. In some embodiments, the length of each segment is about 25 nucleotides to about 10000 nucleotides. In some embodiments, the length of each segment is about 30 nucleotides to about 10000 nucleotides. In some embodiments, the length of each segment is about 35 nucleotides to about 10000 nucleotides. In some embodiments, the length of each segment is about 40 nucleotides to about 10000 nucleotides. In some embodiments, the length of each segment is about 45 nucleotides to about 10000 nucleotides. In some embodiments, the length of each segment is about 50 nucleotides to about 10000 nucleotides. In some embodiments, the length of each segment is about 100 nucleotides to about 10000 nucleotides. In some embodiments, the length of each segment is about 500 nucleotides to about 10000 nucleotides. In some embodiments, the length of each segment is about 1000 nucleotides to about 10000 nucleotides. In some embodiments, the length of each segment is about 5000 nucleotides to about 10000 nucleotides. In some embodiments, the length of each segment is about 15 nucleotides to about 5000 nucleotides. In some embodiments, the length of each segment is about 15 nucleotides to about 1000 nucleotides. In some embodiments, the length of each segment is about 15 nucleotides to about 500 nucleotides. In some embodiments, the length of each segment is about 15 nucleotides to about 100 nucleotides. In some embodiments, the length of each segment is about 15 nucleotides to about 50 nucleotides. In some embodiments, the length of each segment is about 15 nucleotides to about 45 nucleotides. In some embodiments, the length of each segment is about 15 nucleotides to about 40 nucleotides. In some embodiments, the length of each segment is about 15 nucleotides to about 35 nucleotides. In some embodiments, the length of each segment is about 15 nucleotides to about 30 nucleotides. In some embodiments, the length of each segment is about 15 nucleotides to about 25 nucleotides. In some embodiments, the length of each segment is about 15 nucleotides to about 20 nucleotides. In some embodiments, the length of each segment is about 40 nucleotides.

In some embodiments, each segment is separated by a spacer. In some embodiments, the length of the spacer is about 0 nucleotide to about 5000 nucleotides. In some embodiments, the length of the spacer is about 10 nucleotide to about 5000 nucleotides. In some embodiments, the length of the spacer is about 50 nucleotide to about 5000 nucleotides. In some embodiments, the length of the spacer is about 100 nucleotide to about 5000 nucleotides. In some embodiments, the length of the spacer is about 500 nucleotide to about 5000 nucleotides. In some embodiments, the length of the spacer is about 1000 nucleotide to about 5000 nucleotides. In some embodiments, the length of the spacer is about 0 nucleotide to about 1000 nucleotides. In some embodiments, the length of the spacer is about 0 nucleotide to about 500 nucleotides. In some embodiments, the length of the spacer is about 0 nucleotide to about 100 nucleotides. In some embodiments, the length of the spacer is about 0 nucleotide to about 50 nucleotides. In some embodiments, the length of the spacer is about 0 nucleotide to about 10 nucleotides.

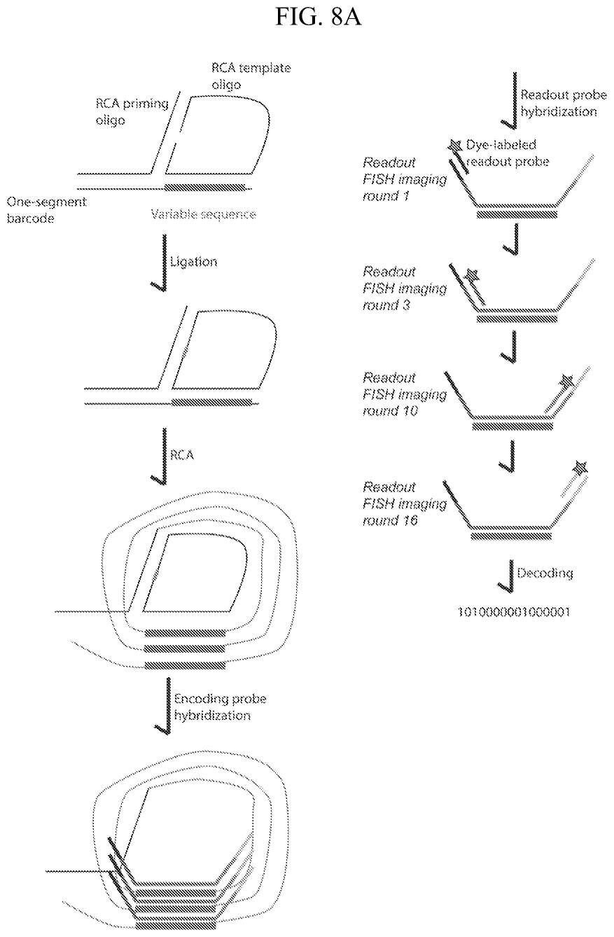

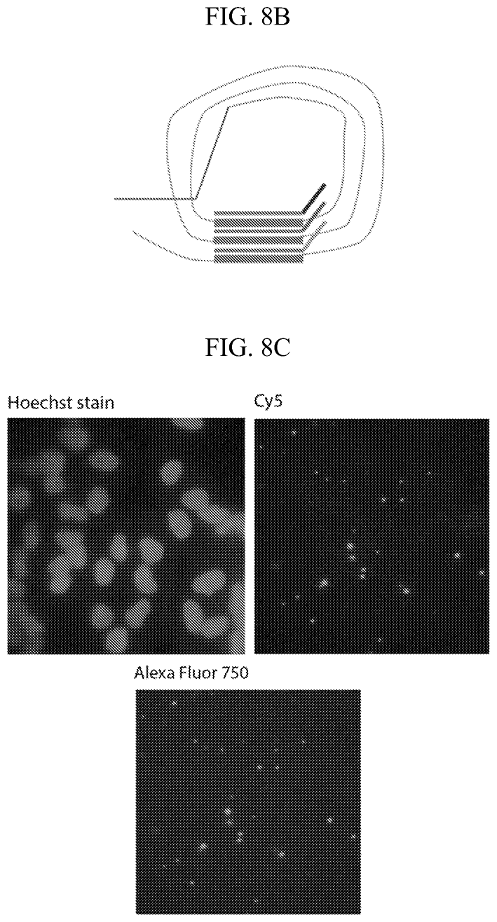

Although nucleic acid barcodes with multiple segments are known in the art, it is possible to design a nucleic acid barcode with only one segment that is still highly functional with methods described herein. Instead of having a readout probe be attached directly to the rolling circle amplification product, the encoding probe comprising one or more unique readout regions binds to the amplification product instead. The readout probes now bind to one of the unique readout regions of the encoding probe, allowing fast and accurate identification of a decoded sequence. An example of this method is shown in FIG. 8.

In some embodiments, a method of decoding a nucleic acid barcode in situ in a sample is provided herein, wherein the method comprises the steps of: a) amplifying at least a target region of a nucleic acid barcode by rolling circle amplification to generate amplified nucleic acids comprising copies of the target region, wherein the target region comprises a primary variable sequence; b) contacting the sample with a plurality of encoding probes under conditions that allow hybridization of said encoding probes to said amplified nucleic acids, wherein at least one of said encoding probe comprises a sequence complementary to a sequence in said amplified nucleic acids, and each said encoding probe comprises one or more of a plurality of unique readout regions; c) contacting the sample with one or more labeled readout probes under conditions that allow hybridization of said labeled readout probes to said encoding probe(s), wherein each said labeled readout probe comprises a sequence complementary to a unique readout region; d) detecting the label(s) of the one or more labeled readout probes; e) optionally eliminating signal from the label(s) of the readout probes detectable in step (d); f) optionally repeating steps (c), (d) and (e) with one or more additional labeled readout probes, each comprising a sequence complementary to a different unique readout region; and g) determining, based on the presence of the one or more labeled readout probes, the identity of the nucleic acid barcode.

In some embodiments, the nucleic acid barcode comprises only one target region. In some embodiments, at least one of said encoding probes comprises a sequence complementary to the primary variable sequence. In some embodiments, each encoding probe comprises two or more of a plurality of unique readout regions. In some embodiments, each encoding probe comprises one of a plurality of unique readout regions. In some embodiments, the nucleic acid barcode is from a library of nucleic acid barcodes.

In some embodiments, step (a) of any of the methods described above comprises: a1) contacting the sample with a pair of oligonucleotide probes under conditions that allow hybridization of said oligonucleotide probes to their respective target sequences, said pair of oligonucleotide probes comprising: (i) a padlock probe comprising at least a region that is complementary to a first part of the nucleic acid barcode, wherein the padlock probe comprises a 5′ end region and 3′ end region and wherein upon said hybridization the 5′ and 3′ end regions of the padlock probe are brought into juxtaposition for circularization of the padlock probe, and wherein when circularized the padlock probe comprises the reverse complementary sequence of the target region; and (ii) a linear probe comprising a region that is complementary to at least a region of the padlock probe; a2) circularizing the padlock probe to form a circular padlock probe; and a3) amplifying the circular padlock probe in situ to generate the amplified nucleic acids comprising copies of the target region.

In some embodiments, the linear probe comprises a binding region and an overhang region, wherein the binding region is complementary to a second part of the nucleic acid barcode and the overhang region is complementary to at least a region of the padlock probe. In some embodiments, the 5′ and 3′ end regions of the padlock probe are hybridized to the overhang region of the linear probe. In some embodiments, the 5′ and 3′ end regions of the padlock probe are hybridized to the first part of the nucleic acid barcode. In some embodiments, the linear probe does not comprise a binding region that is complementary to any part of the nucleic acid barcode.

In some embodiments, when the nucleic acid barcode is from a library of nucleic acid barcodes, the second part of each nucleic acid barcode comprises a unique sequence. In some embodiments, when the nucleic acid barcode is from a library of nucleic acid barcodes, the second part of each nucleic acid barcode comprises the same sequence.

In some embodiments, the nucleic acid barcode does not comprise a second part. In some embodiments, upon padlock probe hybridization, the 5′ and 3′ end regions of the padlock probe are immediately next to each other, and wherein the circularizing step is performed by ligating the 5′ and 3′ end regions of the padlock probe with a ligase. In some embodiments, upon padlock probe hybridization, the 5′ and 3′ end regions of the padlock probe have a gap of one or more nucleotides between each other, and wherein the circularizing step is performed by gap filling with a polymerase or a reverse transcriptase and ligating the 5′ and 3′ end regions of the padlock probe with a ligase. In some embodiments, when the 5′ and 3′ end regions of the padlock probe are hybridized to the first part of the nucleic acid barcode, the length of the gap is shorter than the length of the target region. In some embodiments, when the 5′ and 3′ end regions of the padlock probe are hybridized to the first part of the nucleic acid barcode, the length of the gap is the same or longer than the length of the target region.

In some embodiments, the padlock probe further comprises a secondary variable sequence, wherein said secondary variable sequence is matched with the primary variable sequence, and wherein secondary variable sequence is also amplified during rolling circle amplification. In some embodiments, at least one of said encoding probes comprises a sequence complementary to the secondary variable sequence.

In some embodiments, a method of decoding a nucleic acid barcode in situ in a sample is provided, wherein the nucleic acid barcode comprises only one target region, said target region comprises a primary variable sequence, said method comprising the steps of: a) contacting the sample with a pair of oligonucleotide probes under conditions that allow hybridization of said oligonucleotide probes to their respective target sequences, said pair of oligonucleotide probes comprising: (i) a padlock probe comprising a 5′ end region, a 3′ end region, and a binding region that is complementary to a first part of the nucleic acid barcode, wherein said first part comprises the target region of the nucleic acid barcode; and (ii) a linear probe comprising a binding region and an overhang region, wherein the binding region is complementary to the second part of the nucleic acid barcode, wherein the 5′ end and 3′ end regions of the padlock probe are hybridized to the overhang region of the linear probe and wherein upon said hybridization the 5′ and 3′ end regions of the padlock probe are brought into juxtaposition for circularization of the padlock probe; b) circularizing the padlock probes to form circular padlock probes; c) amplifying the circular padlock probe in situ to generate amplified nucleic acids comprising copies of the target region; d) contacting the sample with a plurality of encoding probes under conditions that allow hybridization of said encoding probes to said amplified nucleic acids, wherein at least one of said encoding probe comprises a sequence complementary to the primary variable sequence, and each said encoding probe comprises one or more of a plurality of unique readout regions; e) contacting the sample with one or more labeled readout probes under conditions that allow hybridization of said labeled readout probes to said encoding probe(s), wherein each said labeled readout probe comprises a sequence complementary to a unique readout region; f) detecting the label(s) of the one or more labeled readout probes; g) optionally eliminating signal from the label(s) of the readout probe(s) detectable in step (f); h) optionally repeating steps (e), (f) and (g) with one or more additional labeled readout probes, each comprising a sequence complementary to a different unique readout region; and i) determining, based on the presence of the one or more labeled readout probes, the identity of the nucleic acid barcode.

In some embodiments, each encoding probe comprises two or more of a plurality of unique readout regions. In some embodiments, each encoding probe comprises one of a plurality of unique readout regions.

In some embodiments, the nucleic acid barcode is from a library of nucleic acid barcodes. In some embodiments, the second part of each nucleic acid barcode comprises a unique sequence. In some embodiments, the second part of each nucleic acid barcode comprises the same sequence.

In some embodiments, the number of unique readout regions is about 2 to about 6000. In some embodiments, the number of unique readout regions is about 5 to about 6000. In some embodiments, the number of unique readout regions is about 10 to about 6000. In some embodiments, the number of unique readout regions is about 50 to about 6000. In some embodiments, the number of unique readout regions is about 100 to about 6000. In some embodiments, the number of unique readout regions is about 500 to about 6000. In some embodiments, the number of unique readout regions is about 1000 to about 6000. In some embodiments, the number of unique readout regions is about 5000 to about 6000. In some embodiments, the number of unique readout regions is about 2 to about 5000. In some embodiments, the number of unique readout regions is about 2 to about 1000. In some embodiments, the number of unique readout regions is about 2 to about 500. In some embodiments, the number of unique readout regions is about 2 to about 100. In some embodiments, the number of unique readout regions is about 2 to about 50. In some embodiments, the number of unique readout regions is about 2 to about 10. In some embodiments, the number of unique readout regions is about 2 to about 5.

In some embodiments, the length of the variable sequence is about 15 nucleotides to about 300 nucleotides. In some embodiments, the length of the variable sequence is about 50 nucleotides to about 300 nucleotides. In some embodiments, the length of the variable sequence is about 100 nucleotides to about 300 nucleotides. In some embodiments, the length of the variable sequence is about 250 nucleotides to about 300 nucleotides. In some embodiments, the length of the variable sequence is about 15 nucleotides to about 250 nucleotides. In some embodiments, the length of the variable sequence is about 15 nucleotides to about 100 nucleotides. In some embodiments, the length of the variable sequence is about 15 nucleotides to about 50 nucleotides. In some embodiments, the length of the variable sequence is about 20 nucleotides.

In some embodiments, the linear probe comprises single-stranded DNA. In some embodiments, the padlock probe comprises single-stranded DNA. In some embodiments, the linear probe comprises single-stranded LNA or single-stranded DNA with partial LNA modification(s). In some embodiments, the padlock probe comprises single-stranded LNA or single-stranded DNA with partial LNA modification(s).

In some embodiments, the linear and padlock probes are added simultaneously. In some embodiments, the linear and padlock probes are added sequentially. In some embodiments, the linear and padlock probes are added simultaneously to the sample. In some embodiments, the linear and padlock probes are added sequentially to the sample.

In some embodiments, the amplification step is performed with a rolling circle amplification DNA polymerase. In some embodiments, the rolling circle amplification DNA polymerase is Phi29 polymerase. In some embodiments, the rolling circle amplification DNA polymerase is Bst polymerase. In some embodiments, the rolling circle amplification DNA polymerase is Vent exo-DNA polymerase.

In some embodiments, the amplification step is performed with a rolling circle amplification RNA polymerase. In some embodiments, the rolling circle amplification RNA polymerase is T7 RNA polymerase.

In some embodiments, the circularization step comprises ligation with a ligase. In some embodiments, the ligase is a DNA ligase. In some embodiments, the DNA ligase is a T4 DNA ligase. In some embodiments, the DNA ligase is a T7 DNA ligase. In some embodiments, the DNA ligase is a T3 DNA ligase. In some embodiments, the DNA ligase is a Taq DNA ligase. In some embodiments, the ligase is an Ampligase. In some embodiments, the DNA ligase is an E. coli DNA ligase. In some embodiments, the ligase is a SplintR ligase.

In some embodiments, the circularization of the padlock probe is performed in situ. In some embodiments, the circularization of the padlock probe is performed in vitro prior to contacting the sample with the padlock probe.

In some embodiments, the readout probes are labeled with fluorescent dyes. In some embodiments, at least some readout probes are labeled with the same fluorescent dye. In some embodiments, at least some readout probes are labeled with different fluorescent dyes. In some embodiments, the fluorescence signal is eliminated by photobleaching, chemical bleaching, chemical cleavage, chemical wash, heat denaturation, nuclease treatment, or a combination thereof. In some embodiments, the fluorescence signal is retained.

In some embodiments, the amplified nucleic acids are crosslinked to the sample. In some embodiments, the crosslinking is performed by aminoallyl-dUTP spike-in during the amplification step, and post-fixation with paraformaldehyde and/or PEGylated bis(sulfosuccinimidyl)suberate (BS(PEG)5 or BS(PEG)9).

In some embodiments, the nucleic acid barcode is comprised in a DNA, RNA, locked nucleic acid (LNA), DNA with partial LNA modification(s), or peptide nucleic acid (PNA) molecule. In some embodiments, the nucleic acid molecule is double-stranded. In some embodiments, the nucleic acid molecule is single-stranded. In some embodiments, the nucleic acid barcode is delivered into cells. In some embodiments, the barcode is not delivered into cells but is decoded on the surface of cells or independent of cells. In some embodiments, the barcode is decoded at a molecular level, cellular level, or multi-cellular level.

Any of the methods described above pertain to the amplification and readout of barcodes in situ. Such methods can be used by themselves, or in a wide number of screens, which are also described herein.

In some embodiments, a method of performing an in situ genetic screen is provided, the method comprising: pairing a genetic screen technique with nucleic acid barcodes; performing the genetic screen technique; and decoding the nucleic acid barcodes with any decoding method described herein.

In some embodiments, the genetic screen technique is a pooled genetic screen technique. In some embodiments, the genetic screen technique is a CRISPR screen technique. In some embodiments, the CRISPR screen is a CRISPR knockout screen. In some embodiments, the CRISPR screen is a CRISPR interference (CRISPRi) screen. In some embodiments, the CRISPR screen is a CRISPR activation (CRISPRa) screen. In some embodiments, the CRISPR screen is a CRISPR screen of cis-regulatory elements. In some embodiments, the CRISPR screen is a CRISPR screen of protein domain functions. In some embodiments, the CRISPR screen is a CRISPR double-perturbation screen. In some embodiments, the genetic screen technique is an RNA interference (RNAi) screen technique. In some embodiments, the genetic screen technique is a massively parallel reporter assay screen.

In some embodiments, the step of pairing a genetic screen technique with nucleic acid barcodes further comprises pairing at least one nucleic acid barcode with at least one nucleic acid genetic perturbation sequence. In some embodiments, each barcode pairs with a unique genetic perturbation sequence. In some embodiments, each barcode and genetic perturbation sequence pairing are located on one polynucleotide sequence. In some embodiments, the nucleic acid barcode is attached to the genetic perturbation sequence. In some embodiments, each barcode and genetic perturbation sequence pairing are located on multiple polynucleotide sequences. In some embodiments, the nucleic acid barcode is the genetic perturbation sequence. In some embodiments, the step of pairing a genetic screen technique with nucleic acid barcodes comprises pairing at least one nucleic acid barcode with a combination of at least two nucleic acid genetic perturbation sequences. In some embodiments, the genetic perturbation sequence is a guide RNA (gRNA). In some embodiments, each genetic perturbation sequence is a unique gRNA. In some embodiments, the nucleic acid barcodes and nucleic acid genetic perturbation sequences are delivered into cells.

In some embodiments, the nucleic acid barcodes and nucleic acid genetic perturbation sequences are delivered into cells by viral transduction. In some embodiments, viral transduction is performed by a lentivirus or an adeno-associated virus (AAV). In some embodiments, the nucleic acid barcodes and nucleic acid genetic perturbation sequences are delivered into cells by transfection. In some embodiments, the nucleic acid barcodes and nucleic acid genetic perturbation sequences are delivered into cells by electroporation. In some embodiments, the nucleic acid barcodes and nucleic acid genetic perturbation sequences are delivered into cells by microinjection.

In some embodiments, the method further comprises analyzing the results of the genetic screen technique to determine a phenotypic perturbation. In some embodiments, the phenotypic perturbation is a perturbation of cell size, cell shape, cellular interactions, cellular abundance/density of biomolecules, sub-cellular abundance/density of biomolecules, sub-cellular morphologies, sub-cellular distribution, and/or sub-cellular organization. In some embodiments, the phenotypic perturbation is a perturbation of genome architecture. In some embodiments, the phenotypic perturbation is a perturbation of three-dimensional chromatin organization.

In some embodiments, the analysis of the results of the genetic screen technique is performed by an imaging technique. In some embodiments, the imaging technique is in situ hybridization. In some embodiments, the imaging technique is fluorescence in situ hybridization. In some embodiments, the imaging technique is multiplexed DNA or RNA fluorescence in situ hybridization. In some embodiments, the imaging technique is imaging of lipid modifications. In some embodiments, the imaging technique is imaging of sugar modifications. In some embodiments, the imaging technique is imaging of metabolite modifications. In some embodiments, the imaging technique is imaging of DNA modifications. In some embodiments, the imaging technique is imaging of RNA modifications. In some embodiments, the imaging technique is imaging of DNA/RNA/protein modifications. In some embodiments, the imaging technique is imaging of lipid, sugar, metabolite, DNA, RNA, protein and/or DNA/RNA/protein modifications, or any combination thereof.

In some embodiments, the method further comprises the step of matching the decoded nucleic acid barcodes with the determined phenotypic perturbation. In some embodiments, the matching of the decoded barcode with the phenotypic perturbation allows for the determination of which genetic perturbation sequence matches which phenotypic perturbation. In some embodiments, the step of analyzing the results of the genetic screen technique to determine a phenotypic perturbation can be performed prior to, during, or after the decoding step.

In some embodiments, a method of performing an in situ genetic screen is provided, the method comprising: creating at least one unique pairing of at least one nucleic acid barcode with at least one nucleic acid genetic perturbation sequence; introducing at least one unique pairing of the at least one barcode and the at least one perturbation sequence to a cell; incubating the cell under conditions that allow the at least one perturbation sequence to cause the cell to display at least one phenotypic perturbation; analyzing the cell by an imaging technique to determine the at least one phenotypic perturbation; decoding the at least one nucleic acid barcode with any decoding method described herein; and determining the at least one genetic perturbation sequence that causes the cell to display the at least one phenotypic perturbation.

Any of the methods described above can also be used to determine the positions of a cell or cells. In some embodiments, those cells are in a tissue or other sample. For example, in single-cell sequencing techniques, genetic or other molecular information about single cells can be attained, but the process typically dissociates those cells from their three-dimensional substrate. By using the barcoding methods described herein, the cells can be identified prior to single-cell sequencing, thus preserving information about their positioning.

In some embodiments, a method of determining cellular positions in a single-cell sequencing is provided, the method comprising: introducing at least one nucleic acid barcode to at least one cell; imaging the at least one cell to determine cellular position; decoding the nucleic acid barcodes with any decoding method described herein; dissociating the at least one cell from its substrate; performing single-cell sequencing on the at least one cell to determine at least the sequence of the nucleic acid barcode associated with the at least one cell; and mapping the at least one cell to the cellular position.

In some embodiments, at least one nucleic acid barcode is comprised in a DNA, RNA, locked nucleic acid (LNA), DNA with partial LNA modification(s), or peptide nucleic acid (PNA) molecule. In some embodiments, at least one nucleic acid barcode is delivered by a viral vector. In some embodiments, the viral vector is a lentivirus or adeno-associated virus (AAV).

In some embodiments, the method comprises introducing a plurality of nucleic acid barcodes to a plurality of cells, wherein each nucleic acid barcode is only present in one cell. In some embodiments, each nucleic acid barcode is a unique nucleic acid barcode. In some embodiments, the at least one cell is present in at least one tissue.

In some embodiments, the step of performing single-cell sequencing on the at least one cell further determines additional genomic information about the at least one cell. In some embodiments, the step of performing single-cell sequencing on the at least one cell further determines the gene expression of the at least one cell. In some embodiments, the step of performing single-cell sequencing on the at least one cell further determines epigenetic/epigenomic information about the at least one cell. In some embodiments, the step of mapping the at least one cell to the cellular position provides spatial-omic information about the at least one cell.

### EMBODIMENTS

Also provided herein are the following non-limiting embodiments.
1. A method of decoding a nucleic acid barcode in situ in a sample, comprising the following steps:
a) amplifying at least a target region of a segment of the nucleic acid barcode by rolling circle amplification to generate amplified nucleic acids comprising copies of the segment target region, wherein the segment target region comprises one of a plurality of unique primary decoder sequences;
b) contacting the sample with one or more labeled readout probes under conditions that allow hybridization of said labeled readout probes to said amplified nucleic acids, wherein each said labeled readout probe comprises a sequence complementary to a sequence in said amplified nucleic acids;
c) detecting the label(s) of the one or more labeled readout probes; and
d) determining, based on the presence and/or identity of the labeled readout probe, the identity of the nucleic acid barcode.
2. The method of embodiment 1, wherein each said labeled readout probe comprises a sequence complementary to one of the plurality of unique primary decoder sequences.
3. The method of embodiment 1 or 2, wherein the nucleic acid barcode comprises a plurality of segments, and each segment comprises a target region comprising one of a unique plurality of unique primary decoder sequences, and wherein
step (a) comprises amplifying at least a target region of each segment of the nucleic acid barcode to generate a set of amplified nucleic acids,
step (b) comprises contacting the sample with one or more labeled readout probes under conditions that allow hybridization of said labeled readout probes to said amplified nucleic acids amplified from a first segment,
step (d) comprises determining, based on the presence and/or identity of the labeled readout probe from each segment, the identity of the nucleic acid barcode, and
wherein the method further comprises the following steps after step (c) and prior to step (d):
e) optionally eliminating signal from the label(s) of the readout probe detectable in step (c); and
f) repeating steps (b), (c), and (e) until the presence and/or identity of the labeled readout probe has been determined for each segment.
4. The method of any of embodiments 1-3, wherein step (a) comprises the following steps:
a1) contacting the sample with a pair of oligonucleotide probes under conditions that allow hybridization of said oligonucleotide probes to their respective target sequences, said pair of oligonucleotide probes comprising:
(i) a padlock probe comprising at least a region that is complementary to a first part of the segment of the nucleic acid barcode, wherein the padlock probe comprises a 5′ end region and 3′ end region and wherein upon said hybridization the 5′ and 3′ end regions of the padlock probe are brought into juxtaposition for circularization of the padlock probe, and wherein when circularized the padlock probe comprises the reverse complementary sequence of the segment target region; and
(ii) a linear probe comprising a region that is complementary to at least a region of the padlock probe;
a2) circularizing the padlock probe to form a circular padlock probe; and
a3) amplifying the circular padlock probe in situ to generate the amplified nucleic acids comprising copies of the segment target region.
5. The method of embodiment 4, wherein the linear probe comprises a binding region and an overhang region, wherein the binding region is complementary to a second part of the segment of the nucleic acid barcode and the overhang region is complementary to the at least a region of the padlock probe.
6. The method of embodiment 5, wherein when the nucleic acid barcode comprises a plurality of segments, the second part of each segment comprises a unique sequence.
7. The method of embodiment 5, wherein when the nucleic acid barcode comprises a plurality of segments, the second part of each segment comprises the same sequence.
8. The method of embodiment 7, wherein the linear probe used for each segment comprises the same sequence.
9. The method of any one of embodiments 5-8, wherein the 5′ and 3′ end regions of the padlock probe are hybridized to the overhang region of the linear probe.
10. The method of any one of embodiments 4-8, wherein the 5′ and 3′ end regions of the padlock probe are hybridized to the first part of the segment of the nucleic acid barcode.
11. The method of embodiment 4 or 10, wherein the linear probe does not comprise a binding region that is complementary to any part of the nucleic acid barcode.
12. The method of any one of embodiments 4, 10, and 11, wherein the segment does not comprise a second part.
13. The method of any one of embodiments 4-12, wherein upon padlock probe hybridization, the 5′ and 3′ end regions of the padlock probe are immediately next to each other, and wherein the circularizing step is performed by ligating the 5′ and 3′ end regions of the padlock probe with a ligase.
14. The method of any one of embodiments 4-12, wherein upon padlock probe hybridization, the 5′ and 3′ end regions of the padlock probe have a gap of one or more nucleotides between each other, and wherein the circularizing step is performed by gap filling with a polymerase or a reverse transcriptase and ligating the 5′ and 3′ end regions of the padlock probe with a ligase.
15. The method of embodiment 14, wherein when the 5′ and 3′ end regions of the padlock probe are hybridized to the first part of the segment of the nucleic acid barcode, the length of the gap is shorter than the length of the target region.
16. The method of embodiment 14, wherein when the 5′ and 3′ end regions of the padlock probe are hybridized to the first part of the segment of the nucleic acid barcode, the length of the gap is the same or longer than the length of the target region.
17. The method of any one of embodiments 4-16, wherein the padlock probe further comprises one of a plurality of unique secondary decoder sequences, wherein each said unique secondary decoder sequence is matched with one of the plurality of unique primary decoder sequences, and wherein the unique secondary decoder sequence is also amplified during rolling circle amplification.
18. The method of embodiment 17, wherein each said labeled readout probe comprises a sequence complementary to one of the plurality of unique secondary decoder sequences.
19. A method of decoding a nucleic acid barcode in situ in a sample, wherein each nucleic acid barcode comprises a plurality of segments, each segment comprising a target region comprises one of a unique plurality of unique primary decoder sequences, the method comprising the following steps:
a) contacting the sample with a plurality of pairs of oligonucleotide probes under conditions that allow hybridization of said pairs of oligonucleotide probes to their respective target sequences in each segment, each said pair of oligonucleotide probes comprising:
   (i) a padlock probe comprising a 5′ end region, a 3′ end region, and a binding region that is complementary to a first part of the segment of the nucleic acid barcode, wherein said first part of the segment comprises a target region of a segment of the nucleic acid barcode and said segment target region comprises one of a plurality of unique primary decoder sequences; and
   (ii) a linear probe comprising a binding region and an overhang region, wherein the binding region is complementary to the second part of the segment, wherein the 5′ end and 3′ end regions of the padlock probe are hybridized to the overhang region of the linear probe and wherein upon said hybridization the 5′ and 3′ end regions of the padlock probe are brought into juxtaposition for circularization of the padlock probe;
b) circularizing the padlock probes to form circular padlock probes;
c) amplifying the circular padlock probes in situ to generate a set of amplified nucleic acids comprising copies of the segment target region;
d) contacting the sample with one or more labeled readout probes under conditions that allow hybridization of said labeled readout probes to said amplified nucleic acids amplified from a first segment, wherein each said labeled readout probe comprises a sequence complementary to one of the plurality of the unique primary decoder sequences in the first segment target region;
e) detecting the label(s) of the one or more labeled readout probes;
f) optionally eliminating signal from the label(s) of the readout probe detectable in step (e);
g) repeating steps (d), (e), and (f) until the presence and/or identity of the labeled readout probe has been determined for each segment; and
h) determining, based on the presence and/or identity of the labeled readout probe from each segment, the identity of the nucleic acid barcode.
20. The method of any one of embodiments 1-19, wherein the barcode comprises 1 to 100 segments.
21. The method of embodiments 20, wherein the barcode comprises about 10 segments.
22. The method of any one of embodiments 1-21, wherein the number of unique primary decoder sequences for each segment is about 2 to about 10000.
23. The method of any one of embodiments 1-22, wherein the number of unique primary decoder sequences for each segment is 3.
24. The method of any one of embodiments 1-23, wherein the length of each segment is about 15 nucleotides to about 10000 nucleotides.
25. The method of any one of embodiments 1-24, wherein the length of each segment is about 40 nucleotides.
26. The method of any one of embodiments 1-25, wherein each segment is separated by a spacer.
27. The method of embodiment 26, wherein the length of the spacer is about 0 nucleotide to about 5000 nucleotides.
28. A method of decoding a nucleic acid barcode in situ in a sample, comprising the following steps:
a) amplifying at least a target region of a nucleic acid barcode by rolling circle amplification to generate amplified nucleic acids comprising copies of the target region, wherein the target region comprises a primary variable sequence;
b) contacting the sample with a plurality of encoding probes under conditions that allow hybridization of said encoding probes to said amplified nucleic acids, wherein at least one of said encoding probe comprises a sequence complementary to a sequence in said amplified nucleic acids, and each said encoding probe comprises one or more of a plurality of unique readout regions;
c) contacting the sample with one or more labeled readout probes under conditions that allow hybridization of said labeled readout probes to said encoding probe(s), wherein each said labeled readout probe comprises a sequence complementary to a unique readout region;
d) detecting the label(s) of the one or more labeled readout probes;
e) optionally eliminating signal from the label(s) of the readout probes detectable in step (d);
f) optionally repeating steps (c), (d) and (e) with one or more additional labeled readout probes, each comprising a sequence complementary to a different unique readout region; and
g) determining, based on the presence of the one or more labeled readout probes, the identity of the nucleic acid barcode.
29. The method of embodiment 28, wherein the nucleic acid barcode comprises only one target region.
30. The method of embodiment 28 or 29, wherein at least one of said encoding probes comprises a sequence complementary to the primary variable sequence.
31. The method of any of embodiments 28-30, wherein each encoding probe comprises two or more of a plurality of unique readout regions.
32. The method of any of embodiments 28-30, wherein each encoding probe comprises one of a plurality of unique readout regions.
33. The method of any of embodiments 28-30, wherein the nucleic acid barcode is from a library of nucleic acid barcodes.
34. The method of any of embodiments 28-33, wherein step (a) comprises the following steps:
a1) contacting the sample with a pair of oligonucleotide probes under conditions that allow hybridization of said oligonucleotide probes to their respective target sequences, said pair of oligonucleotide probes comprising:
   (i) a padlock probe comprising at least a region that is complementary to a first part of the nucleic acid barcode, wherein the padlock probe comprises a 5′ end region and 3′ end region and wherein upon said hybridization the 5′ and 3′ end regions of the padlock probe are brought into juxtaposition for circularization of the padlock probe, and wherein when circularized the padlock probe comprises the reverse complementary sequence of the target region; and
   (ii) a linear probe comprising a region that is complementary to at least a region of the padlock probe;
a2) circularizing the padlock probe to form a circular padlock probe; and
a3) amplifying the circular padlock probe in situ to generate the amplified nucleic acids comprising copies of the target region.
35. The method of embodiment 34, wherein the linear probe comprises a binding region and an overhang region, wherein the binding region is complementary to a second part of the nucleic acid barcode and the overhang region is complementary to at least a region of the padlock probe.
36. The method of embodiment 35, wherein the 5′ and 3′ end regions of the padlock probe are hybridized to the overhang region of the linear probe.
37. The method of embodiment 34 or 35, wherein the 5′ and 3′ end regions of the padlock probe are hybridized to the first part of the nucleic acid barcode.
38. The method of embodiment 34 or 37, wherein the linear probe does not comprise a binding region that is complementary to any part of the nucleic acid barcode.
39. The method of any of embodiments 34-38, wherein when the nucleic acid barcode is from a library of nucleic acid barcodes, the second part of each nucleic acid barcode comprises a unique sequence.
40. The method of any of embodiments 34-38, wherein when the nucleic acid barcode is from a library of nucleic acid barcodes, the second part of each nucleic acid barcode comprises the same sequence.
41. The method of any one of embodiments 34, 37, and 38, wherein the nucleic acid barcode does not comprise a second part.
42. The method of any one of embodiments 34-41, wherein upon padlock probe hybridization, the 5′ and 3′ end regions of the padlock probe are immediately next to each other, and wherein the circularizing step is performed by ligating the 5′ and 3′ end regions of the padlock probe with a ligase.
43. The method of any one of embodiments 34-41, wherein upon padlock probe hybridization, the 5′ and 3′ end regions of the padlock probe have a gap of one or more nucleotides between each other, and wherein the circularizing step is performed by gap filling with a polymerase or a reverse transcriptase and ligating the 5′ and 3′ end regions of the padlock probe with a ligase.
44. The method of embodiment 43, wherein when the 5′ and 3′ end regions of the padlock probe are hybridized to the first part of the nucleic acid barcode, the length of the gap is shorter than the length of the target region.
45. The method of embodiment 43, wherein when the 5′ and 3′ end regions of the padlock probe are hybridized to the first part of the nucleic acid barcode, the length of the gap is the same or longer than the length of the target region.
46. The method of any one of embodiments 34-45, wherein the padlock probe further comprises a secondary variable sequence, wherein said secondary variable sequence is matched with the primary variable sequence, and wherein secondary variable sequence is also amplified during rolling circle amplification.
47. The method of embodiment 46, wherein at least one of said encoding probes comprises a sequence complementary to the secondary variable sequence.
48. A method of decoding a nucleic acid barcode in situ in a sample, wherein the nucleic acid barcode comprises only one target region, said target region comprises a primary variable sequence, said method comprising the following steps:
a) contacting the sample with a pair of oligonucleotide probes under conditions that allow hybridization of said oligonucleotide probes to their respective target sequences, said pair of oligonucleotide probes comprising:
   (i) a padlock probe comprising a 5′ end region, a 3′ end region, and a binding region that is complementary to a first part of the nucleic acid barcode, wherein said first part comprises the target region of the nucleic acid barcode; and
   (ii) a linear probe comprising a binding region and an overhang region, wherein the binding region is complementary to the second part of the nucleic acid barcode, wherein the 5′ end and 3′ end regions of the padlock probe are hybridized to the overhang region of the linear probe and wherein upon said hybridization the 5′ and 3′ end regions of the padlock probe are brought into juxtaposition for circularization of the padlock probe;
b) circularizing the padlock probes to form circular padlock probes;
c) amplifying the circular padlock probe in situ to generate amplified nucleic acids comprising copies of the target region;
d) contacting the sample with a plurality of encoding probes under conditions that allow hybridization of said encoding probes to said amplified nucleic acids, wherein at least one of said encoding probe comprises a sequence complementary to the primary variable sequence, and each said encoding probe comprises one or more of a plurality of unique readout regions;
e) contacting the sample with one or more labeled readout probes under conditions that allow hybridization of said labeled readout probes to said encoding probe(s), wherein each said labeled readout probe comprises a sequence complementary to a unique readout region;
f) detecting the label(s) of the one or more labeled readout probes;
g) optionally eliminating signal from the label(s) of the readout probe(s) detectable in step (f);
h) optionally repeating steps (e), (f) and (g) with one or more additional labeled readout probes, each comprising a sequence complementary to a different unique readout region; and
i) determining, based on the presence of the one or more labeled readout probes, the identity of the nucleic acid barcode.
49. The method of embodiment 48, wherein each encoding probe comprises two or more of a plurality of unique readout regions.
50. The method of embodiment 48, wherein each encoding probe comprises one of a plurality of unique readout regions.
51. The method of any of embodiments 48-50, wherein the nucleic acid barcode is from a library of nucleic acid barcodes.
52. The method of embodiment 51, wherein the second part of each nucleic acid barcode comprises a unique sequence.
53. The method of embodiment 51, wherein the second part of each nucleic acid barcode comprises the same sequence.
54. The method of any one of embodiments 28-53, wherein the number of unique readout regions is about 2 to 6000.
55. The method of any one of embodiments 28-54, wherein the length of the variable sequence is about 15 nucleotides to about 300 nucleotides.
56. The method of embodiment 55, wherein the length of the variable sequence is about 20 nucleotides.
57. The method of any one of embodiments 4-27 and 34-56, wherein the linear and/or padlock probes are single-stranded DNA.
58. The method of any one of embodiments 4-27 and 34-56, wherein the linear and/or padlock probes are single-stranded LNA or single-stranded DNA with partial LNA modification(s).
59. The method of any one of embodiments 4-27 and 34-58, wherein the linear and padlock probes are added simultaneously.
60. The method of any one of embodiments 4-27 and 34-58, wherein the linear and padlock probes are added sequentially.
61. The method of any one of embodiments 1-60, wherein the amplification step is performed with a rolling circle amplification DNA polymerase.
62. The method of embodiment 61, wherein the rolling circle amplification DNA polymerase is Phi29, Bst, or Vent exo-DNA polymerase.
63. The method of any one of embodiments 1-60, wherein the amplification step is performed with a rolling circle amplification RNA polymerase.
64. The method of embodiment 63, wherein the rolling circle amplification RNA polymerase is T7 RNA polymerase.
65. The method of any one of embodiments 4-27 and 34-64, wherein the circularization step comprises ligation with a ligase.
66. The method of embodiment 65, wherein the ligase is a DNA ligase.
67. The method of embodiment 66, wherein DNA ligase is a T4 DNA ligase, T7 DNA ligase, T3 DNA ligase, Taq DNA ligase, Ampligase, or E. coli DNA ligase.
68. The method of embodiment 65, wherein the ligase is a SplintR ligase.
69. The method of any one of embodiments 4-27 and 34-68, wherein the circularization of the padlock probe is performed in situ.
70. The method of any one of embodiments 4-27 and 34-68, wherein the circularization of the padlock probe is performed in vitro prior to contacting the sample with the padlock probe.
71. The method of any one of embodiments 1-70, wherein the readout probes are labeled with fluorescent dyes.
72. The method of embodiment 71, wherein at least some readout probes are labeled with the same fluorescent dye.
73. The method of embodiment 71, wherein at least some readout probes are labeled with different fluorescent dyes.
74. The method of any one of embodiments 3-73, wherein the fluorescence signal is eliminated by photobleaching, chemical bleaching, chemical cleavage, chemical wash, heat denaturation, nuclease treatment, or a combination thereof
75. The method of any one of embodiments 3-73, wherein the fluorescence signal is retained.
76. The method of any one of embodiments 1-75, wherein the amplified nucleic acids are crosslinked to the sample.
77. The method of embodiment 76, wherein the crosslinking is performed by aminoallyl-dUTP spike-in during the amplification step, and post-fixation with paraformaldehyde and/or PEGylated bis(sulfosuccinimidyl)suberate (BS(PEG)5 or BS(PEG)9).
78. The method of any of embodiments 1-77, wherein the nucleic acid barcode is comprised in a DNA, RNA, locked nucleic acid (LNA), DNA with partial LNA modification(s), or peptide nucleic acid (PNA) molecule.
79. The method of embodiment 78, wherein the nucleic acid molecule is double-stranded.
80. The method of embodiment 78, wherein the nucleic acid molecule is single-stranded.
81. The method of any of embodiments 1-80, wherein the nucleic acid barcode is delivered into cells.
82. The method of any of embodiments 1-80, wherein the barcode is not delivered into cells but is decoded on the surface of cells or independent of cells.
83. The method of any of embodiments 1-82, wherein the barcode is decoded at a molecular level, cellular level, or multi-cellular level.
84. A method of performing an in situ genetic screen, comprising the following steps:
pairing a genetic screen technique with nucleic acid barcodes;
performing the genetic screen technique; and
decoding the nucleic acid barcodes with a decoding method of any of embodiments 1-83.
85. The method of embodiment 84, wherein the genetic screen technique is a pooled genetic screen technique.
86. The method of embodiments 84 or 85, wherein the genetic screen technique is a CRISPR screen technique.
87. The method of embodiment 86, wherein the CRISPR screen is a CRISPR knockout screen, a CRISPR interference (CRISPRi) screen, a CRISPR activation (CRISPRa) screen, a CRISPR screen of cis-regulatory elements, a CRISPR screen of protein domain functions, or a CRISPR double-perturbation screen.
88. The method of embodiments 84 or 85, wherein the genetic screen technique is an RNA interference (RNAi) screen technique.
89. The method of embodiments 84 or 85, wherein the genetic screen technique is a massively parallel reporter assay screen.
90. The method of any of embodiments 84-89, wherein the step of pairing a genetic screen technique with nucleic acid barcodes further comprises pairing at least one nucleic acid barcode with at least one nucleic acid genetic perturbation sequence.
91. The method of embodiment 90, wherein each barcode pairs with a unique genetic perturbation sequence.
92. The method of any of embodiments 90-91, wherein each barcode and genetic perturbation sequence pairing are located on one polynucleotide sequence.
93. The method of any of embodiments 90-91, wherein the nucleic acid barcode is attached to the genetic perturbation sequence or is the genetic perturbation sequence.
94. The method of any of embodiments 90-91, wherein each barcode and genetic perturbation sequence pairing are located on multiple polynucleotide sequences.
95. The method of any of embodiments 90-94, wherein the step of pairing a genetic screen technique with nucleic acid barcodes comprises pairing at least one nucleic acid barcode with a combination of at least two nucleic acid genetic perturbation sequences.
96. The method of any of embodiments 90-95, wherein the genetic perturbation sequence is a guide RNA (gRNA).
97. The method of any of embodiments 90-96, wherein each genetic perturbation sequence is a unique gRNA.
98. The method of any of embodiments 90-97, wherein the nucleic acid barcodes and nucleic acid genetic perturbation sequences are delivered into cells.
99. The method of any of embodiments 90-98, wherein the nucleic acid barcodes and nucleic acid genetic perturbation sequences are delivered into cells by viral transduction, transfection, electroporation, or microinjection.
100. The method of any of embodiments 90-99, wherein the nucleic acid barcodes and nucleic acid genetic perturbation sequences are delivered into cells by viral transduction.
101. The method of embodiment 100, wherein viral transduction is performed by a lentivirus or an adeno-associated virus (AAV).
102. The method of any of embodiments 90-101, wherein the method further comprises analyzing the results of the genetic screen technique to determine a phenotypic perturbation.
103. The method of embodiment 102, wherein the phenotypic perturbation is a perturbation of cell size, cell shape, cellular interactions, cellular abundance/density of biomolecules, sub-cellular abundance/density of biomolecules, sub-cellular morphologies, sub-cellular distribution, and/or sub-cellular organization.
104. The method of embodiments 102 or 103, wherein the phenotypic perturbation is a perturbation of genome architecture.
105. The method of embodiment 104, wherein the phenotypic perturbation is a perturbation of three-dimensional chromatin organization.
106. The method of any of embodiments 102-105, wherein the analysis of the results of the genetic screen technique is performed by an imaging technique.
107. The method of embodiment 106, wherein the imaging technique is in situ hybridization.
108. The method of embodiments 106 or 107, wherein the imaging technique is fluorescence in situ hybridization.
109. The method of any of embodiments 106-108, wherein the imaging technique is multiplexed DNA or RNA fluorescence in situ hybridization.
110. The method of embodiment 106, wherein the imaging technique is imaging of lipid, sugar, metabolite, DNA, RNA, protein and/or DNA/RNA/protein modifications.
111. The method of any of embodiments 102-110, wherein the method further comprises the step of matching the decoded nucleic acid barcodes with the determined phenotypic perturbation.
112. The method of embodiment 111, wherein the matching of the decoded barcode with the phenotypic perturbation allows for the determination of which genetic perturbation sequence matches which phenotypic perturbation.
113. The method of any of embodiments 102-112, wherein the step of analyzing the results of the genetic screen technique to determine a phenotypic perturbation can be performed prior to, during, or after the decoding step.
114. A method of performing an in situ genetic screen, comprising the following steps:
creating at least one unique pairing of at least one nucleic acid barcode with at least one nucleic acid genetic perturbation sequence;
introducing at least one unique pairing of the at least one barcode and the at least one perturbation sequence to a cell;
incubating the cell under conditions that allow the at least one perturbation sequence to cause the cell to display at least one phenotypic perturbation;
analyzing the cell by an imaging technique to determine the at least one phenotypic perturbation;
decoding the at least one nucleic acid barcode with a decoding method of any of embodiments 1-83; and
determining the at least one genetic perturbation sequence that causes the cell to display the at least one phenotypic perturbation.
115. A method of determining cellular positions in a single-cell sequencing, comprising the following steps:
introducing at least one nucleic acid barcode to at least one cell;
imaging the at least one cell to determine cellular position;
decoding the nucleic acid barcodes with a decoding method of any of embodiments 1-83;
dissociating the at least one cell from its substrate;
performing single-cell sequencing on the at least one cell to determine at least the sequence of the nucleic acid barcode associated with the at least one cell; and
mapping the at least one cell to the cellular position.
116. The method of embodiment 115, wherein the at least one nucleic acid barcode is comprised in a DNA, RNA, locked nucleic acid (LNA), DNA with partial LNA modification(s), or peptide nucleic acid (PNA) molecule.
117. The method of embodiments 115 or 116, wherein the at least one nucleic acid barcode is delivered by a viral vector.
118. The method of embodiment 117, wherein the viral vector is a lentivirus or adeno-associated virus (AAV).
119. The method of any of embodiments 115-118, wherein the method comprises introducing a plurality of nucleic acid barcodes to a plurality of cells, and wherein each nucleic acid barcode is only present in one cell.
120. The method of embodiment 119, wherein each nucleic acid barcode is a unique nucleic acid barcode.
121. The method of any of embodiments 115-120, wherein the at least one cell is present in at least one tissue.
122. The method of any of embodiments 115-121, wherein the step of performing single-cell sequencing on the at least one cell further determines additional genomic information about the at least one cell.
123. The method of any of embodiments 115-122, wherein the step of performing single-cell sequencing on the at least one cell further determines the gene expression of the at least one cell.
124. The method of any of embodiments 115-123, wherein the step of performing single-cell sequencing on the at least one cell further determines epigenetic/epigenomic information about the at least one cell.
125. The method of any of embodiments 115-124, wherein the step of mapping the at least one cell to the cellular position provides spatial-omic information about the at least one cell.

### EXAMPLES

The following examples are illustrative, but not limiting, of the methods described herein.

### Example 1: Pooled In Situ Barcoding with Rolling Circle Amplification and Fluorescence In Situ Hybridization (BARC-FISH 1)

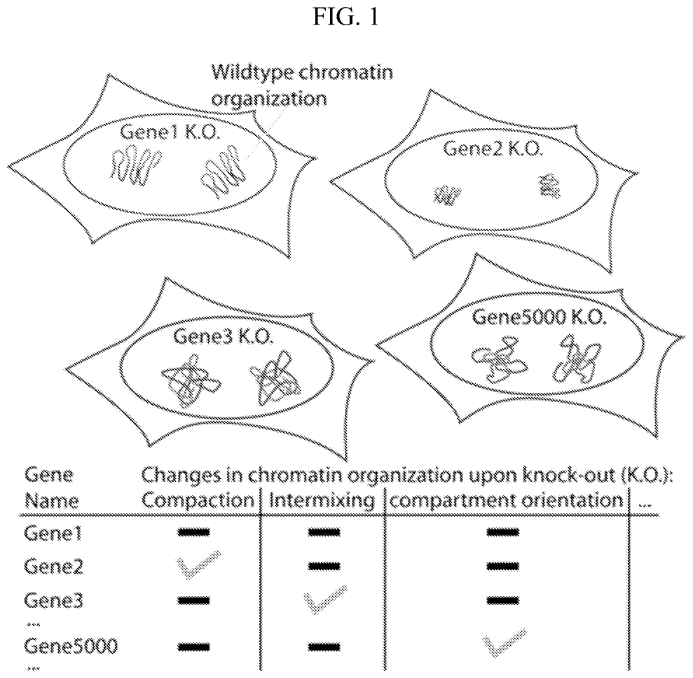

Pooled genetic screens, such as CRISPR or RNAi screens, are powerful methods to discover genetic factors that affect various biomedical processes. Current pooled genetic screens are largely focused on growth or gene expression phenotypes. For example, a pool of cells with various genetic perturbations undergoes selection (through the biomedical process or by cell sorting), and the remaining cells are sequenced to reveal the perturbations enriched/depleted through the selection. Pooled genetic screen techniques that allow robust, high-throughput, and flexible screening of factors regulating in situ phenotypes are lacking. The in situ phenotypes include cell size, cell shape, cellular interactions, cellular abundance/density of biomolecules, sub-cellular abundance/density of biomolecules, sub-cellular morphologies of cellular compartments and organelles, sub-cellular distribution and organization of biomolecules. For example, genome architectures such as chromatin folding patterns are important in situ phenotypes. Correct three-dimensional (3D) organization of chromatin is essential for the proper functioning of cells in the human body. Defective chromatin organization can alter cellular behavior and is a hallmark of aging and multiple diseases including cancer and progeria, among others. Despite its critical importance, understanding how the 3D organization of chromatin is regulated at the molecular level in health and disease remains a major challenge for the scientific and biomedical community. FIG. 1 diagrams a concept of an image-based pooled screen of chromatin organization regulators. So far, no technique has been demonstrated to robustly and efficiently screen for regulators of chromatin folding, while such a method is desperately needed.

### Methods

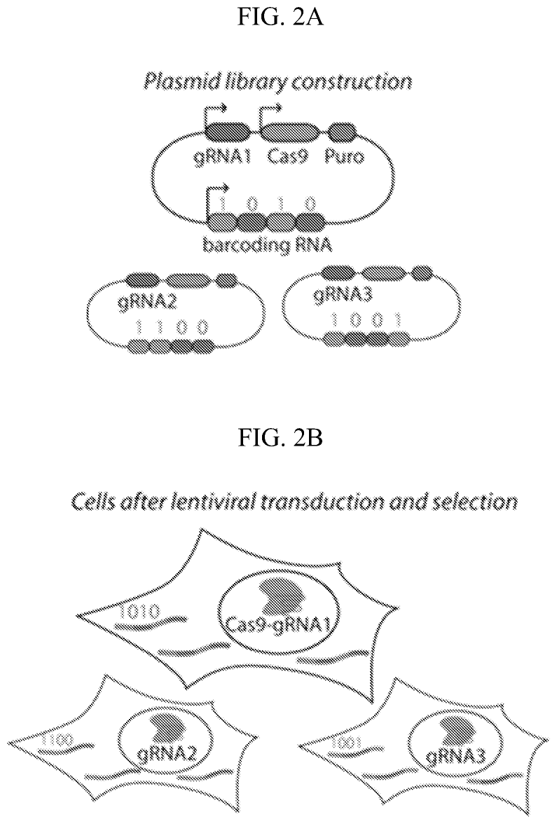

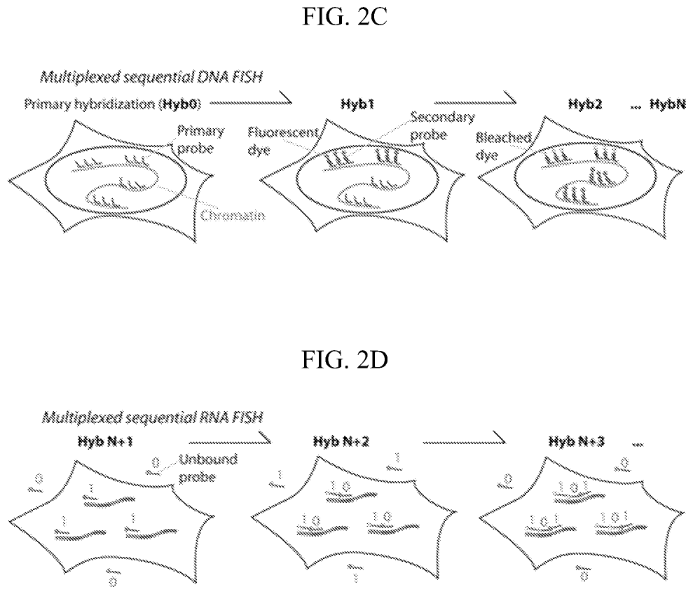

A new in situ barcoding method is described herein. The method is combined with multiplexed DNA and RNA fluorescence in situ hybridization (FISH), as well as CRISPR screen techniques, to allow pooled genetic screen of regulators of in situ phenotypes, including chromatin folding. In short, various barcode regions were constructed in a pool of plasmids carrying CRISPR guide RNAs (gRNAs) that introduce genetic perturbations into cells (FIG. 2A). The barcode regions are expressed into RNAs in cells during the screen (FIG. 2B), and can be amplified through oligonucleotide probe hybridization, ligation, and rolling circle amplification (RCA) (FIG. 3). Then the barcodes can be read out and distinguished from each other with multiplexed RNA FISH (FIGS. 2D and 3). This barcoding and readout procedure is termed Barcode Amplification by Rolling Circle and readout by FISH (BARC-FISH). The various barcodes are paired with various gRNAs in the plasmids, so that each barcode RNA uniquely maps to a gRNA in the screen. In other words, BARC-FISH allows a user to identify the genetic perturbations in situ in a screen (FIGS. 2A-B). To determine the chromatin organization in the cells, multiplexed DNA FISH (chromatin tracing, ref 10) was applied to the same cells either before, after, or during the BARC-FISH procedure (FIG. 2C). In this way, genetic perturbations that lead to chromatin folding changes can be identified. The multiplexed FISH may consist of one or multiple rounds of sequential readout FISH hybridization. In each round of readout hybridization, one or multiple versions of fluorescence-labeled readout probes in one or multiple fluorescence colors are hybridized to the sample to read out the barcode digit(s) or pinpoint the genomic locus (loci) (ref 11, 12). After imaging the fluorescence, the fluorescence may be eliminated (by photobleaching (ref 11, 12), chemical bleaching, chemical cleavage (ref 13), chemical wash (ref 14), heat denaturation, nuclease treatment (ref 15), or their combinations) before the next round of sequential readout hybridization and imaging.

Notably, the application of BARC-FISH is not limited to in situ CRISPR screen or screen for chromatin folding regulators. It is broadly applicable to situations where cells need to be barcoded and decoded in situ. Particularly, BARC-FISH is expected to be compatible with multiple types of genetic screens, including CRISPR screens, RNAi screens, massively parallel reporter assay screens, etc. It is expected to be compatible with different versions of the same type of screen. For example, for CRISPR screen, this invention can be combined with CRISPR knockout screen, CRISPR interference (CRISPRi) screen, CRISPR activation (CRISPRa) screen, CRISPR screen of cis-regulatory elements, CRISPR screen of protein domain functions, CRISPR double-perturbation screens, etc. In all these cases, the invention can enable in situ phenotypic screens.

Cloning of the sgRNA-Barcode Association Library; Timing 3-4 Days

PCR amplification of barcode and sgRNA segments. The barcode segments are amplified by limited-cycle PCR from a premade barcode plasmid library which contains all of the 10-trit barcodes (3¹⁰=59,049 barcodes by design). Similarly, the sgRNA segments are PCR-amplified from either a premade sgRNA plasmid library or a CustomArray oligo pool. All PCR reactions are performed using Phusion High Fidelity PCR Master Mix (New England BioLabs). The amplified barcode and sgRNA segments are then subject to electrophoresis and spin-column purification using Zymoclean Gel DNA Recovery Kit (Zymo Research).

Restriction digest of plasmid backbone. The plasmids are digested with the restriction enzyme Esp3I (Thermo Fisher) overnight at 37 degrees. In the restriction digest master mix, add Alkaline Phosphatase (Thermo Fisher) to remove the phosphate groups from the DNA ends. The digested products are subject to electrophoresis and spin-column purification.

Gibson Assembly of barcode, sgRNA and plasmid backbone. The purified barcode segments, sgRNA segments and the plasmid backbone are mixed together at a molar ratio of 10:10:1 with Gibson Assembly Master Mix. The mixture is incubated at 50 degrees for 1 hr.

Purification of Gibson products by isopropanol precipitation. After the Gibson Assembly, the reaction products are purified by isopropanol precipitation. Briefly, mix the products with 50% isopropanol, 50 mM sodium chloride and 0.075 μg/μl GlycoBlue Coprecipitant (Thermo Fisher). Then the mixture is incubated for 15 min and centrifuged at ˜15,000 g for 15 min at room temperature to precipitate the DNA. The DNA pellet is rinsed twice with 1 mL of ice-cold 80% ethanol and dissolved in TE buffer for the subsequent bacterial transformation.

Bacterial transformation of sgRNA-barcode association library. 100 ng library is introduced into Endura electrocompetent cells (Lucigen) by electroporation following the manufacturer's instructions. The electroporated cells are recovered by shaking at ˜225 rpm, 37 degrees for 1 hr. Then the liquid culture is spread onto the LB agar plates containing 100 μg/mL ampicillin, and incubated at 37 degrees overnight.

Harvest of plasmid DNA. After the overnight growth, 1,000-2,000 bacterial colonies are collected from the plates and cultured in 200 mL LB liquid medium overnight by shaking at ˜225 rpm, 37 degrees. The plasmid DNA is extracted and purified by maxi-prep using QIAGEN EndoFree Plasmid Maxi Kit.

### Construction of Mammalian CRISPR Screen Library; Timing 2-3 Weeks

Production of lentivirus library. The sgRNA-barcode plasmid library harvested from the previous step is transfected into HEK 293FT cells to make the lentivirus library. Briefly, the plasmid library and helper plasmids psPAX2 (Addgene #12260) and pVSV-G (Addgene #138479) are mixed with Lipofectamine 2000 (Thermo Fisher) following the manufacturer's instructions, and the mixture is added into the cell culture. 2 days after the lentiviral transfection, the lentivirus supernatant is collected from the cell culture, and cell debris is removed by filtering through 0.45 μm strainer.

Lentiviral transduction into mammalian cells and resistance selection. A549 lung cancer cells are cultured to −80-90% confluency and co-cultured with the lentivirus supernatant at a series of different titrations. 2 days after the lentiviral transduction, Puromycin is added into the cell culture at 3 μg/mL to select the cells with resistance. The cells are monitored daily and the medium is refreshed with Puromycin when necessary. After 10 to 12 days, the selection process will be completed.

### BARC-FISH; Timing 4-5 Days

Hybridization of BARC-FISH probes. In this step, BARC-FISH is performed to target transcribed RNA molecules that carry the barcodes. Specifically, the A549 cell culture are fixed in freshly made 4% PFA in 1× DPBS for 10 min at RT and washed twice with 1× DPBS. The fixed cells are then permeabilized by 0.5% Triton in 1× DPBS for 10 min at RT. The cells are incubated with pre-hybridization buffer for 5 min at RT. For BARC-FISH using DNA probes, 20% formamide, 0.1% Tween-20 in 2× SSC is used. The formamide concentration is subject to change according to different BARC-FISH designs, where the melting temperature of probes may be different. The cells are then hybridized with 800 nM BARC-FISH probes in the hybridization buffer (20% formamide, 0.1 mg/mL Salmon sperm DNA, 100 nM helper probes and 1% murine RNase inhibitor in 2× SSC) overnight at 37 degrees.

Rolling-circle amplification (RCA). After the overnight probe hybridization, the cells are washed twice with 40% formamide in 2× SSCT for 15 min at RT, and once with 4× SSC in 1× DPBS with 0.1% Tween-20 (DPBSTw) at 37 degrees for 20 min to remove the excessive unbound probes. The formamide concentration and temperature in this washing condition are subject to change according to different BARC-FISH designs. Then incubate the cells with 0.5 U/μL T4 DNA ligase (Thermo Fisher) in the ligation buffer (1× T4 DNA Ligase buffer, 0.2 mg/mL BSA, 1% murine RNase inhibitor, supplemented with 1 mM DTT and 1 mM ATP) for 2 hrs at RT. After the ligation reaction, the cells are briefly washed twice with 1× DPBSTw and incubated with 1 U/μL Phi29 DNA polymerase in reaction buffer (1× Phi29 reaction buffer, 250 μM dNTP, 0.2 mg/mL BSA, 1% murine RNase inhibitor, supplemented with 1 mM DTT) for 4-6 hrs at 30 degrees water bath. The cells are rinsed twice with 1× DPBSTw and post-fix the RCA amplicons with 4% PFA in 1× DPBS for 30 min at RT and washed with 1× DPBS twice.

Phenotype detection. Phenotype detection steps, for example, antibody staining of a protein of interest for studying its subcellular localization, or DNA FISH for detecting chromatin architecture, may be conducted at this step or prior to the hybridization of BARC-FISH probes.

Sequential FISH and imaging for barcode detection. Fiducial beads diluted in 2× SSC are applied to the cell sample for correcting sample drift during multiple rounds of imaging. The cell sample with beads is then assembled into a flow chamber, and mounted onto a microscope with an automated fluidics system. 10 rounds of readout hybridization buffer (20% ethylene carbonate (EC), 3 nM of each readout probes in 2× SSC) are prepared. Each readout buffer contains 3 different dye-labeled readout probes targeting the 3 different values of each digit in the barcode. During each round of sequential FISH and imaging, the cell sample is incubated with the readout hybridization buffer for 30 min at RT. Then the sample is washed with wash buffer (20% EC in 2× SSC) for 2 min and exchange to oxygen-scavenging imaging buffer (2× SSC, 50 mM Tris·HCl (pH 8.0), 10% glucose, 2 mM Trolox, 0.5 mg/mL glucose oxidase and 40 μg/mL catalase). Images are taken in four different color channels across multiple fields of view. After the imaging, the readout probes of the current round are stripped off by washing with bleach buffer (90% formamide, 1× DPBS), followed by a brief washing process with wash buffer to ensure the successful removal of excessive bleach buffer, and the next round of readout hybridization will be introduced. The process is repeated until 10 rounds of sequential FISH and imaging are completed. Phenotype detection steps may be incorporated in the sequential FISH and imaging process, for example, visualizing protein subcellular localization by antibody staining pattern, or visualizing chromatin architecture via sequential FISH.

### Results

### Develop a BARC-FISH Method to Read Out RNA Barcodes in Cell Culture

To design a pooled genetic screen such as CRISPR screen, a proper method to identify phenotypes of interest (phenotyping), and to associate the observed phenotype with the corresponding perturbation (genotyping) is needed. Here, the phenotyping of chromatin architecture is achieved through multiplexed DNA FISH technique termed chromatin tracing and the genotyping is achieved through multiplexed RNA FISH of various barcoding RNAs, each of which is uniquely paired with a guide RNA (gRNA) in the CRISPR library (FIG. 2A). In the original design, the barcode RNAs are directly targeted by dye-labeled readout oligonucleotide probes, with one copy of the probe for each barcode digit (FIG. 2D). Although traditional single-molecule RNA FISH usually requires at least tens of probes to tile a single copy of RNA molecule to generate sufficient signal from the background and sufficient distinction from non-specifically bound probes, here a cell as a whole only needs to have a stronger RNA FISH signal when a barcode digit is expressed, thus with a strong promoter driving the barcode expression, perhaps one probe per barcode digit is enough. However, tests quickly showed that in human cell culture, even with a strong CMV promoter, the “one probe per digit” design does not provide sufficient distinction between the overall FISH signals from a cell expressing the barcode digit versus a cell not expressing the digit. Using a longer digit sequence to allow tens of probes to bind is not feasible, as the overall barcode length would exceed the lentiviral packaging limit in the library delivery by lentiviral transduction (FIG. 2B). A signal amplification scheme is needed to generate the desired distinction.

To amplify the signal and read out each barcode digit with a high signal-to-background ratio, an RCA strategy was adopted to amplify each digit, and then each digit can be visualized by sequential RNA FISH. This strategy is termed Barcode Amplification by Rolling Circle and readout by FISH (BARC-FISH). Specifically, a set of linear probe and padlock probe is hybridized to each digit (FIG. 3). The linear probe consists of a targeting region that hybridizes to half of the digit, and an overhang region. The padlock probe consists of a targeting region that hybridizes to the other half of the digit, and two overhang regions, the ends of which hybridize to the overhang region of the linear probe and are positioned next to each other. The two ends are then ligated, and thus the padlock probe is circularized. Next, the barcode sequence to which the padlock probe is hybridized is amplified by RCA DNA polymerase in situ, generating many copies of this “half-digit”. The “half-digit” can then be effectively visualized by FISH with a high signal-to-background ratio due to its high local concentration (FIG. 3).

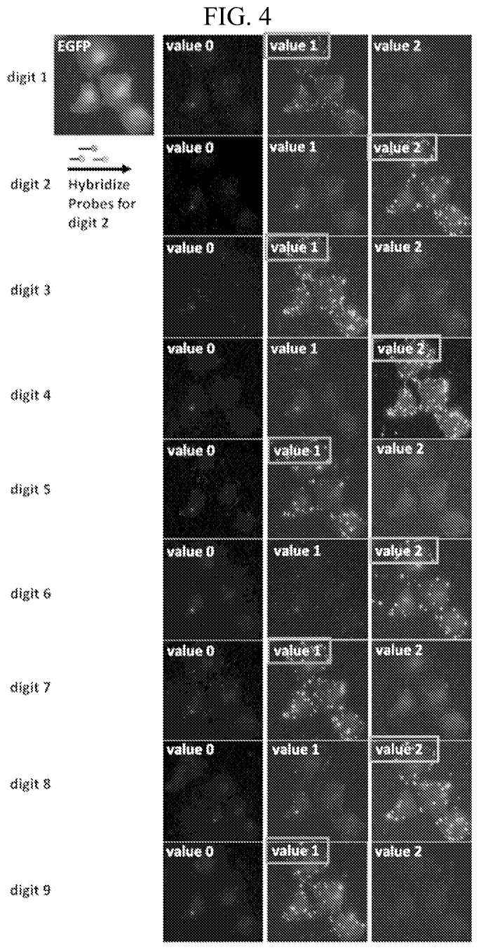

To test whether BARC-FISH can be used to sequentially read out barcodes with high accuracy, a lentivirus vector expressing test barcode #1 was constructed, with a value of 1212121212. All value-1 digits were visualized using Cy5-labeled readout probes in a 647-nm laser illuminated fluorescence channel, and value-2 using Cy7-labeled readout probes in a 750-nm channel. During each round of sequential hybridization, the value-1 and value-2 readout probes for the current digit were simultaneously hybridized to the sample, and the sample was imaged in both the 750-nm and 647-nm channels. Next, the sample was washed with 65% formamide in 2× SSC to remove the current readout probes from the sample (in combination with photobleaching to ensure complete signal removal), and then the readout probes for the next digit were applied. The barcode DNA fragment was commercially synthesized, and inserted downstream of a CMV promoter and EGFP gene. The constructed lentivirus was then transduced into A549 cells. The expectation was that EGFP+ cells should express the test barcode in mRNA form, which can be visualized by BARC-FISH. Indeed, bright BARC-FISH foci were seen in EGFP+ cells in high density in the correct channel after each round of sequential hybridization (FIG. 4). The cells were correctly decoded as carrying a 1212121212 barcode.

### Automatically Decode Distinct Barcodes in a Mixed Cell Culture

To test whether BARC-FISH can distinguish mixed cell populations carrying different barcodes, another construct carrying test barcode #2 was designed, which has value of 2121212121 (every digit has the opposite value to test barcode #1) and is associated with the mCherry mRNA. The lentiviral construct was transduced into A549 cells, and then the EGFP+ cells and mCherry+ cells were mixed, and BARC-FISH was conducted. The expectation was that the EGFP+ cells and mCherry+ cells could be correctly decoded and distinguished based on their barcode values.

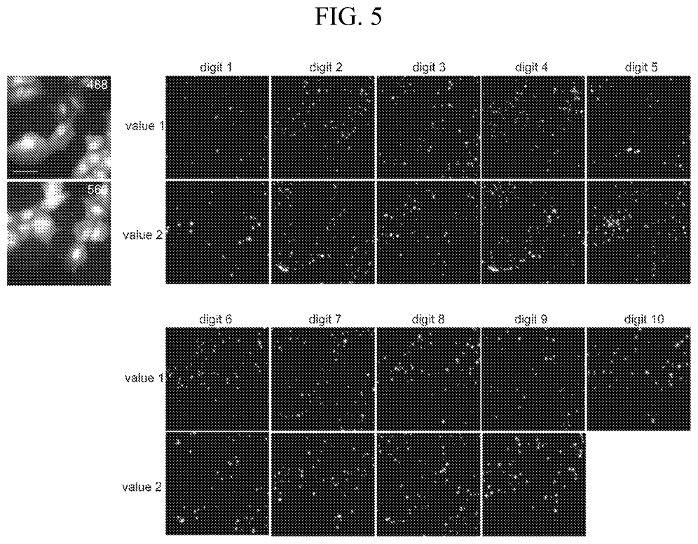

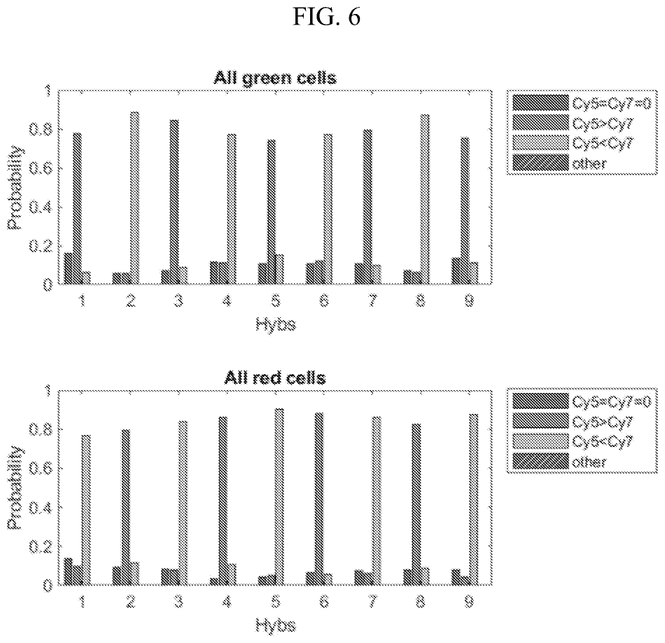

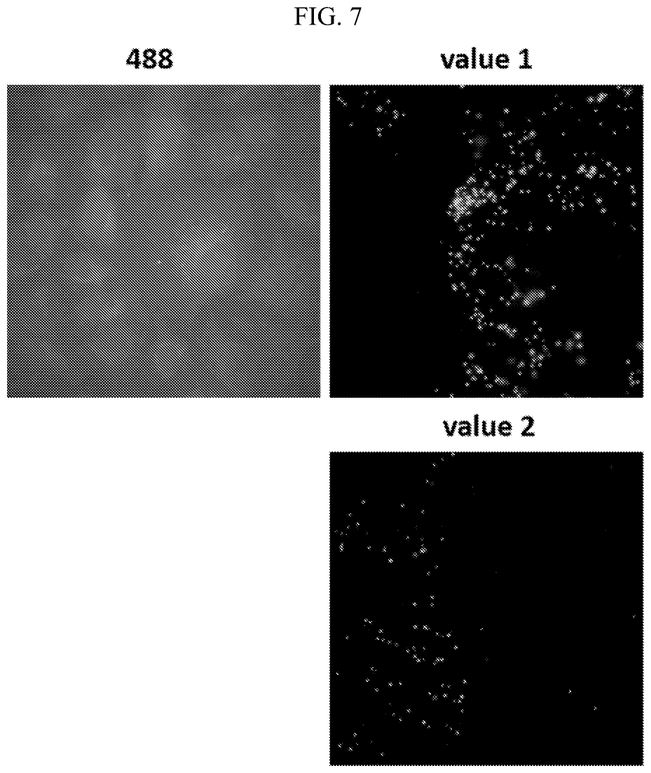

FIG. 5 shows a representative field of view of all digits. In the odd digits (digits 1, 3, 5, . . . ), EGFP+ cells showed strong value-1 BARC-FISH signal (visualized in the 647-nm channel) and mCherry+ cells showed strong value-2 signal (visualized in the 750-nm channel). In the even digits (2, 4, 6, . . . ), mCherry+ cells showed strong value-1 BARC-FISH signal (visualized in the 647-nm channel) and EGFP+ cells showed strong value-2 signal (visualized in the 750-nm channel). Cells and all barcode digits were analyzed in an automated fashion. The cells were computationally segmented by their fluorescence protein pattern and DAPI signal using watershed algorithm, and their true identities were determined by their relative fluorescence intensities of EGFP and mCherry. The computer program then decoded the cells through each digit by comparing the fluorescent pixel counts in the 647-nm and 750-nm channels. If there were more 647-nm pixels than 750-nm pixels in the cell, it was decoded as having value 1 for the current digit; if there were more 750 pixels than 647 pixels, the cell was decoded as having value 2. About 600 EGFP+ cells and ˜700 mCherry+ cells were processed and collected in a BARC-FISH experiment. FIG. 6 shows the correct identification rate of the barcodes. In both mCherry+ cells and EGFP+ cells, most cells were correctly decoded.

Clone a CRISPR-Cas9 Library with Paired Guide RNAs and Barcodes

To test the new screen technique on a small scale, a pooled cloning strategy was adopted to construct a test CRISPR screen library with 420 gRNAs (targeting 136 genes with 3 gRNAs per gene, one gene with 2 gRNAs, and 10 non-targeting control gRNAs) paired with different barcodes. A 10-trit barcode design was used (10 digits with three possible values at each digit, see FIG. 3). To associate gRNAs with barcodes, the DNA fragments of gRNAs were ligated to barcode DNA fragments and inserted into lentiviral vector by Gibson Assembly. The plasmid library was electroporated into E. coli cells, and 3,500 colonies were collected. During the assembly of gRNA-barcode fragment, all gRNAs have the chance of pairing with any of the 3¹⁰ (59,049) different barcode sequences. However, after the colony collection, since the collected colonies were much fewer than the total number of possible barcodes, the chance of one barcode appearing more than once in the collected library and associating with multiple gRNAs is minimized. In other words, the vast majority of the barcodes in the collected library should be uniquely paired with individual gRNAs. Indeed, using next generation sequencing, 87.4% of the barcodes in the collected CRISPR plasmid library were uniquely paired with individual gRNAs, and all gRNAs were about evenly represented in the library (data not shown).

Test DNA Oligonucleotides with LNA Modifications as Linear and Padlock Probes

In the initial tests, the linear and padlock probes are single-stranded DNA oligonucleotides. Recently DNA with partial locked nucleic acid (LNA) modifications was tested for the linear and padlock probes, and showed that these probes also work in BARC-FISH and may have better signal generation efficiency (FIG. 7).

Combination of BARC-FISH with DNA FISH in Chromatin Tracing

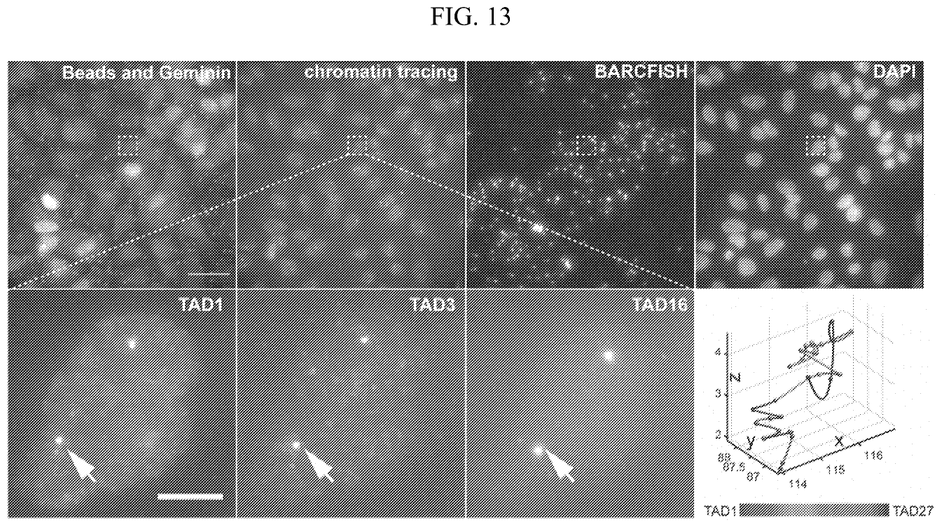

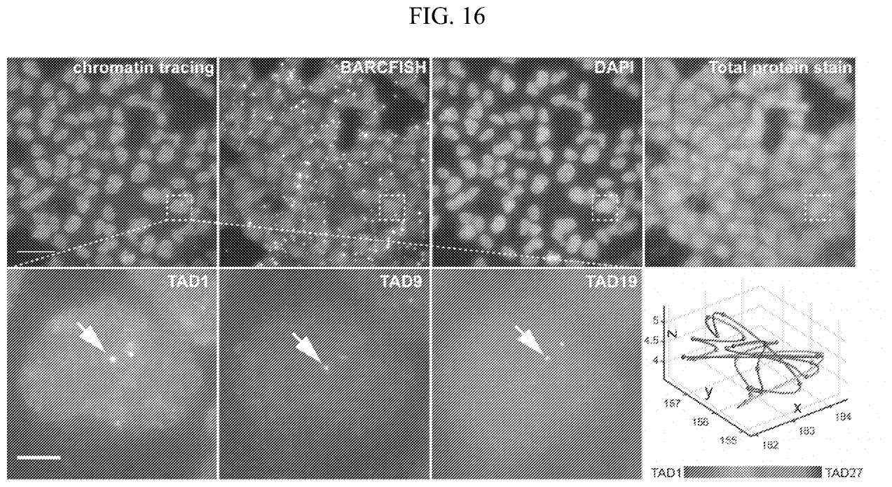

It has been demonstrated that BARC-FISH can be combined with DNA FISH in the chromatin tracing procedure (FIGS. 13 and 16). In the demonstration, barcode hybridization, ligation, and RCA were performed first. During the RCA procedure, aminoallyl-dUTP was optionally spiked into the polymerization reaction. Then the RCA products were crosslinked to the sample by a paraformaldehyde post-fixation and/or reacting with BS(PEG)9. This crosslinking helped better retain the RCA product during the subsequent DNA FISH procedure.

### Example 2: Pooled In Situ Barcoding with Rolling Circle Amplification and Fluorescence In Situ Hybridization Version 2 (BARC-FISH 2)

Another version of BARC-FISH design is described herein and denoted as BARC-FISH 2. In the BARC-FISH 2 design, the barcode consists of one segment of barcode sequence (instead of 10 as demonstrated above), and the number of sequence varieties of this single segment is much larger (e.g. 10,000 instead of 3 as demonstrated above) (FIG. 8A). To distinguish the numerous varieties of this single segment, after RCA, a library of oligonucleotide probes (termed encoding probes) targeting the variable sequences on the RCA products is introduced (FIG. 8A). Each encoding probe will contain a targeting region that hybridizes to the RCA product, and a combination of readout regions that can hybridize to dye-labeled readout probes in subsequent readout FISH hybridization (FIG. 8A). The various barcode sequences are encoded with combinations of the readout regions, and are decoded and distinguished with the subsequent multiplexed readout FISH (FIG. 8A). Alternatively, each encoding probe may have only one (or more) readout region, but multiple encoding probes forming a combination of different readout regions may target the same RCA product (FIG. 8B). This also allows the various barcode sequences to be encoded with combinations of the readout regions, and to be decoded with the subsequent multiplexed readout FISH. In other words, each encoding probe may or may not carry all the readout regions that are used in the combination.

BARC-FISH 2 is performed with similar methodology as BARC-FISH 1, as described above. First, three oligo libraries are designed and synthesized: a gRNA library with each gRNA sequence linked to a unique short barcode sequence, a padlock probe library carrying the reverse complementary sequences of the short barcode sequences, and an encoding probe library. The short barcode segment can be generated by first generating random sequences, then screening for sequences with good melting temperature (which could range from 30-100 degrees Celsius or a subset of this range) and lack of significant homology with each other and with the transcriptome of the target cell. The length of the segment could potentially vary from 10 nt to hundreds of nt. As additional examples, all secondary/readout probe binding sequences in multiplexed FISH studies can potentially be used as barcode segment sequences for BARC-FISH 1 or 2, as these are designed following the same principles. The difference between BARC-FISH 1 and 2 is BARC-FISH 1 requires multiple barcode segments to form the entire barcode; BARC-FISH 2 only needs one segment.

Second, the gRNA library is cloned into a vector (e.g. lentiviral vector) for delivery into cells. Third, fix and permeabilize the cells expressing the barcode as in BARC-FISH 1. Perform the hybridization of the linear probe and the padlock probe (use the padlock probe library). Ligate the ends of each padlock probe. Perform rolling circle amplification primed by the linear probe. Fourth, hybridize the encoding probes to the rolling circle amplicons. The hybridization condition is comparable to the readout hybridization condition in BARC-FISH 1. Fifth and finally, perform sequential readout hybridization and imaging using dye-labeled readout probes (these probes hybridize to the encoding probes). The hybridization condition is comparable to the readout hybridization condition in BARC-FISH 1.

To demonstrate the BARC-FISH 2 design, a single barcode segment in a single-clone cell culture was targeted. The encoding probe carries the code “0110001100000000” (The encoding probe carries readout regions #2, 3, 7 and 8). Then the 7th and 8th digits were determined with readout probes labeled with Cy5 and Alexa Fluor 750 respectively. The fluorescence images showed reoccurring fluorescence foci in both fluorescence channels (FIG. 8C)—demonstrating that each RCA product carries multiple digits and potentially can be used to decode the full barcode.

The BARC-FISH 2 strategy has three advantages in comparison to BARC-FISH 1. First, the entire barcode sequence is much shorter, and thus the molecular assembly, cloning, and delivery of such barcode sequences become easier. Second, the barcode sequences may not have to be generated in addition to the sequences that introduce the genetic perturbation. Instead, the sequences for genetic perturbation may themselves serve as barcodes. For example, in a CRISPR screen, the gRNA region can be targeted with BARC-FISH 2—the gRNA region itself may serve as a barcode region. Consider the following example sequence:

(SEQ ID NO: 1)
gtggaaaggacgaaacaccgAAAAAACGAACGGATTAACAgttttag
agctagaaatagcaagttaaaataaggctagtccgttatcaacttga
aaaagtggcaccgagtcggtgcttttttTCCGTACGGCGCACTTAGC
TTAATGGGAAGGTGAAAGTGTaagcttggcgtaactagatcttga

The capital, bolded and underlined region is the gRNA spacer target sequence; the normal underlined region that follows is the gRNA scaffold. The capital, bolded and italicized region is the barcode segment that will bind to the padlock probe; and the capital italicized region that follows will bind to the linear probe. The normal font regions at both ends of the sequence are homology arms used for PCR amplification and cloning into vectors using isothermal assembly. Alternatively, the padlock probe can bind to the gRNA spacer target sequence; the linear probe can bind to the gRNA scaffold.

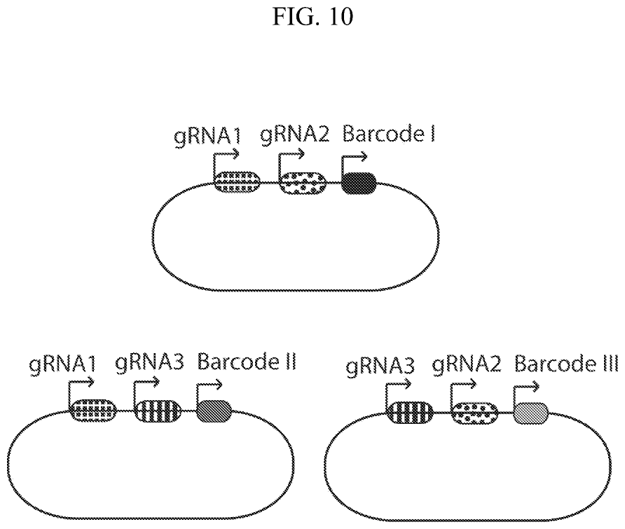

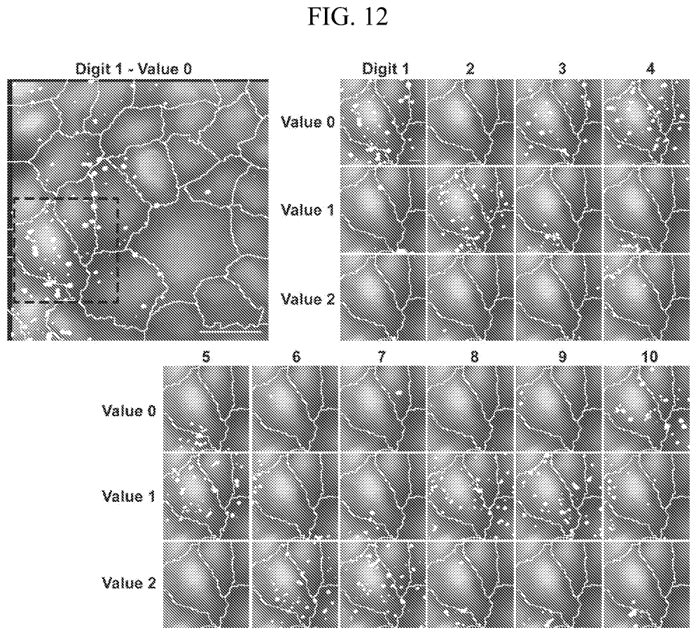

Third, the current protocol for BARC-FISH 1 has low signaling efficiency—the same barcode RNA is rarely detected in more than one round of imaging (FIGS. 4 and 12). In other words, instead of having 10 RCA products for all 10 segments on the same barcode RNA molecule, only one of the segments is usually successfully amplified on each barcode RNA molecule. As a result, in BARC-FISH 1, BARC-FISH signals are aggregating in each whole cell from multiple barcode RNA molecules to decode the full barcode at a cellular level. In the BARC-FISH 2, successful decoding of the full barcode only depends on the successful RCA of one segment, so a barcode from even a single RCA product can be decoded. In other words, decoding is possible at a single-molecule level. This advantage is useful in multiple scenarios. For example, combinations of genetic perturbations can be introduced into the same cells to screen for epistatic relationships/combinatorial effects of the genes/genetic elements. Using BARC-FISH 1, given the currently low signaling efficiency, multiple genetic perturbation sequences (e.g. gRNAs) must be cloned onto the same plasmid to pair each combination with a barcode (see FIG. 10). In BARC-FISH 2, the viral titer can be increased in the viral transduction, so that each cell can receive two or more viral particles. In this case, RCA products carrying different, full barcodes that correspond to different perturbations in the same cell can be decoded.

### Example 3: Different Padlock Probe Types

Signal amplification based on circularization of a padlock probe and rolling circle amplification of the circularized padlock probe has been demonstrated before in in situ sequencing designs. Multiple versions of the circularization designs are expected to be compatible with any version of BARC-FISH. For example, instead of having the ends of the padlock probe hybridized to a linear probe, the ends can be directly hybridized to the barcode segment (FIG. 9A). If the barcode segment is DNA, the ends can be ligated with standard DNA ligase. If the barcode segment is RNA, the ends can be ligated with SplintR ligase, which ligates adjacent single-stranded DNA splinted by RNA. After the ligation, a linear probe hybridized to the padlock probe can serve as the RCA primer. In this design, the linear probe no longer needs to contain a segment that hybridizes to the barcode segment (FIG. 9A).

Furthermore, after the padlock probe hybridization, the ends of the padlock probe could be immediately next to each other, thus ready to be ligated with one of the procedures above. Or, the ends could be multiple nucleotides away from each other, leaving a gap in between, which can be filled with DNA polymerase (when the barcode segment is DNA) or reverse transcriptase (when the barcode segment is RNA) before the ligation reaction (FIG. 9B). The gap could span part of or the entire barcode segment (FIG. 9B).

Another design alternative is: Instead of having the readout probe or encoding probe hybridized to the amplified barcode segment in BARC-FISH, the readout probe or encoding probe can be hybridized to another variable region on the RCA product, derived from a variable region on the padlock probe, as long as this variable region contains different sequences that correspond to the different sequences of the barcode segment (FIG. 9C).

### Example 4: Multiple-Perturbation Genetic Screen Studies with BARC-FISH

In genetic screen studies, after a first screen introducing individual perturbations into individual cells to identify effective genetic elements, it is often desired to perform a secondary screen introducing pairs of (or more) perturbations into the same cells to screen for the perturbations' combined effect and/or to map the epistatic relationship between genes/genetic elements. BARC-FISH can achieve this double-(or more-) perturbation scheme by increasing the viral titer in the transduction so that cells receive a multiplicity of infection (MOI) of more than 1. Alternatively, in both BARC-FISH 1 and 2, each plasmid can carry more than one genetic perturbations, and have the barcoding RNA on each plasmid uniquely map to and indicate the pair of perturbations. For example, FIG. 10 shows a plasmid with two out of three gRNAs in the pool paired with one unique barcode.

### Example 5: Spatial Omics Studies with BARC-FISH

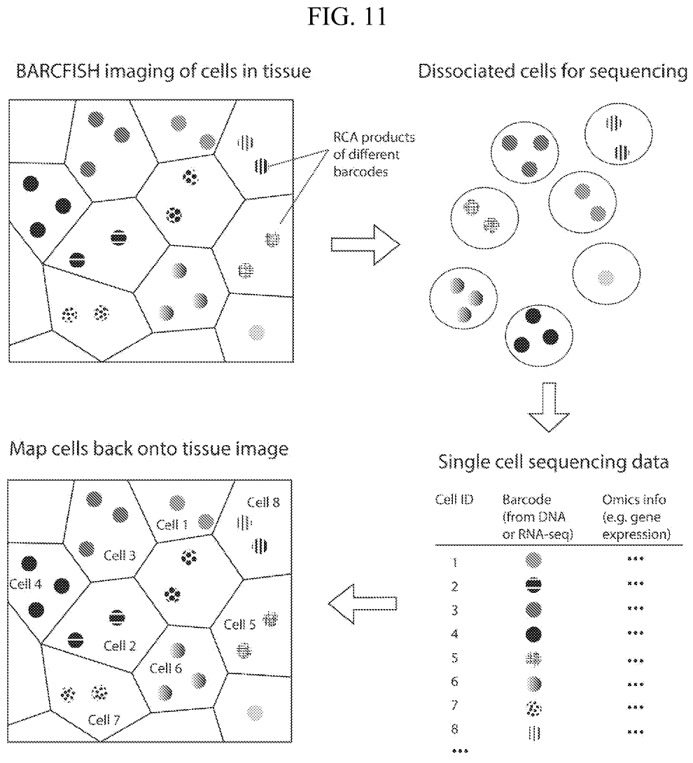

The application of BARC-FISH 1 or 2 is not limited to genetic screens. In one scenario, BARC-FISH may be applied to mark individual cells in space, e.g., to enable spatial omics by combining BARC-FISH and single-cell sequencing. In single-cell sequencing studies, single cells are often first dissociated from their tissue or substrate. As a result, the original spatial position information of the single cells is lost. Using BARC-FISH 1 or 2, one may first deliver the barcodes into individual cells, image the barcodes using BARC-FISH and record the spatial positions of each barcode associated with each cell, dissociate the cells from the tissue/substrate, perform single-cell sequencing in which one also sequences the barcodes (or unique molecular identifiers associated with the barcodes), and then map the sequenced single cells back onto the spatial image by matching the barcodes from sequencing and from BARC-FISH (FIG. 11). This allows one to retain the original spatial information of cells while obtaining their omics information from sequencing.

### Example 6: Imaging-Based Screens Using BARC-FISH 1 and BARC-FISH 2

CRISPR screen libraries were constructed using the BARC-FISH 1 and BARC-FISH 2 designs, respectively. Imaging-based screens were carried out with the libraries.

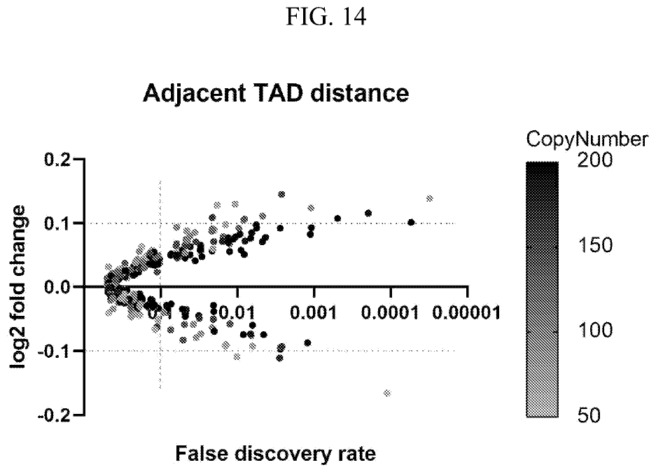

For the BARC-FISH 1 screen, a plasmid library carrying 420 sgRNAs targeting 137 genes was constructed. Each plasmid carries one sgRNA and one 10-digit barcode, with each digit having three possible values (i.e., a 10-trit barcode). The library was then transduced into A549 cells, which constitutively express Cas9 protein, by lentivirus transduction to construct a cell library. The pairing relationship between sgRNAs and barcodes was mapped using next-generation sequencing of the cell library. BARC-FISH 1 was then performed to detect the sgRNA identity in each cell by visualizing and reading out the barcode expressed by the cell (FIG. 12). Specifically, 10 rounds of 3-color sequential FISH hybridization were performed with 30 readout probes to visualize the 10 trits on the barcodes. On the same sample, chromatin tracing (multiplexed DNA FISH) was also performed to detect the folding conformation of human chromosome 22 (chr22) (FIG. 13), together with the imaging of other important molecules and cellular features, including antibody staining of Geminin and nuclear staining with DAPI (FIG. 13). Extensive analyses were performed on various phenotypes related to chromatin conformation upon the different CRISPR perturbations and identified candidate genes regulating each phenotype. For example, candidate genes that regulate the compaction between adjacent topologically associating domains (TADs) on chr22 were identified (FIG. 14).

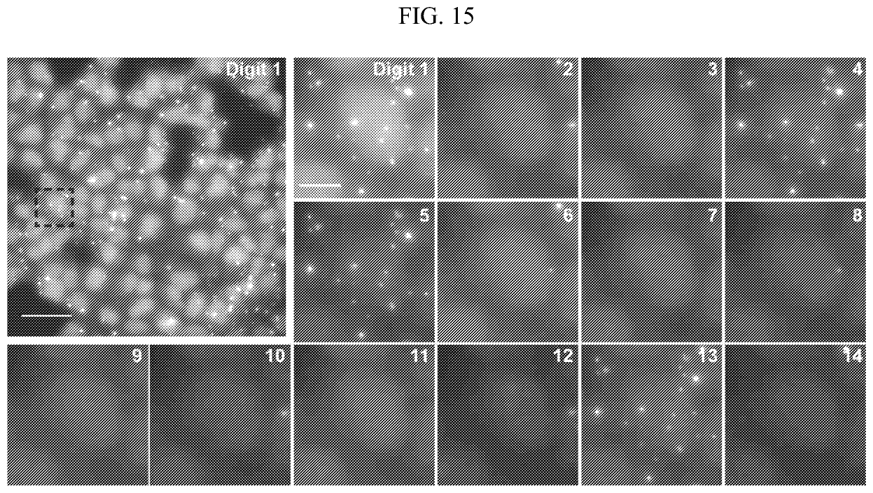

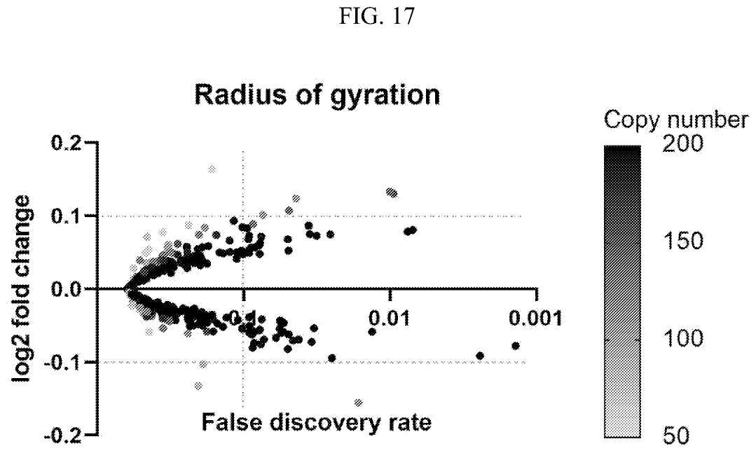

For BARC-FISH 2 screen, two plasmid libraries were constructed, each carrying approximately 500 sgRNAs targeting approximately 500 genes. Each plasmid carries one sgRNA and one single-segment barcode. Two cell libraries were then constructed with lentiviral transduction as in the BARC-FISH 1 screen. BARC-FISH 2 was then performed to detect the sgRNA identity in each cell (FIG. 15) and multi-model imaging similar to BARC-FISH 1, including chromatin tracing, nuclear staining and total protein staining, was performed (FIG. 16). In the BARC-FISH 2 procedure, a set of encoding probes carrying 14-choose-4 combinatorial codes (combinations of 4 readout regions out of 14 total available readout regions) were hybridized to the amplified barcode segment, and the BARC-FISH 2 foci were visualized and decoded via 14 rounds of sequential FISH. As above, various phenotypes were analyzed and candidate genes were identified that regulate the phenotypes, e.g. the radius of gyration of chr22, which is a measurement of overall chromosome compactness (FIG. 17).

### REFERENCES

1 Misteli T. Beyond the sequence: cellular organization of genome function. Cell 2007; 128:787-800. https://doi.org/10.1016/j.cell.2007.01.028.
2 Gorkin D U, Leung D, Ren B. The 3D genome in transcriptional regulation and pluripotency. Cell Stem Cell 2014; 14:762-75. https://doi.org/10.1016/j.stem.2014.05.017.
3 Levine M, Cattoglio C, Tjian R. Looping back to leap forward: transcription enters a new era. Cell 2014; 157:13-25. https://doi.org/10.1016/j.cell.2014.02.009.
4 Dekker J, Mirny L. The 3D Genome as Moderator of Chromosomal Communication. Cell 2016; 164:1110-21. https://doi.org/10.1016/jce11.2016.02.007.
5 Bickmore W A, van Steensel B. Genome architecture: domain organization of interphase chromosomes. Cell 2013; 152:1270-84. https://doi.org/10.1016/j.cell.2013.02.001.
6 Cremer T, Cremer M. Chromosome territories. Cold Spring Harb Perspect Biol 2010; 2:a003889. https://doi.org/10.1101/cshperspect.a003889.
7 Zink D, Fische A H, Nickerson J A. Nuclear structure in cancer cells. Nat Rev Cancer 2004; 4:677-87. https://doi.org/10.1038/nrc1430.
8 Criscione S W, De Cecco M, Siranosian B, Zhang Y, Kreiling J A, Sedivy J M, Neretti N. Reorganization of chromosome architecture in replicative cellular senescence. Sci Adv 2016; 2:e1500882. https://doi.org/10.1126/sciadv.1500882.
9 McCord R P, Nazario-Toole A, Zhang H, Chines P S, Zhan Y, Erdos M R, Collins F S, Dekker J, Cao K. Correlated alterations in genome organization, histone methylation, and DNA-lamin A/C interactions in Hutchinson-Gilford progeria syndrome. Genome Res 2013; 23:260-9. https://doi.org/10.1101/gr.138032.112.
10 Hu M, Wang S. Chromatin Tracing: Imaging 3D Genome and Nucleome. Trends Cell Biol 2021:5-8. https://doi.org/10.1016/j.tcb.2020.10.006.
11 Chen K H, Boettiger A N, Moffitt J R, Wang S, Zhuang X. Spatially resolved, highly multiplexed RNA profiling in single cells. Science 2015; 348: aaa6090. https://doi.org/10.1126/science.aaa6090.
12 Wang S, Su J H, Beliveau B J, Bintu B, Moffitt J R, Wu C, Zhuang X. Spatial organization of chromatin domains and compartments in single chromosomes. Science 2016; 353:598-602. https://doi.org/10.1126/science.aaf8084.
13 Moffitt J R, Hao J, Wang G, Chen K H, Babcock H P, Zhuang X. High-throughput single-cell gene-expression profiling with multiplexed error-robust fluorescence in situ hybridization. Proc Natl Acad Sci USA 2016; 113:11046-51. https://doi.org/10.1073/pnas.1612826113.
14 Takei Y, Yun J, Ollikainen N, Zheng S, Pierson N, White J, Shah S, Thomassie J, Linus Eng C H, Guttman M, Yuan G, Cai L. Global architecture of the nucleus in single cells by DNA seqFISH+ and multiplexed immunofluorescence. BioRxiv 2020:2020.11.29.403055. https://doi.org/10.1101/2020.11.29.403055.
15 Shah S, Lubeck E, Zhou W, Cai L. In Situ Transcription Profiling of Single Cells Reveals Spatial Organization of Cells in the Mouse Hippocampus. Neuron 2016; 92:342-57. https://doi.org/10.1016/j.neuron.2016.10.001.
16 Feldman D, Singh A, Schmid-Burgk J L, Carlson R J, Mezger A, Garrity A J, Zhang F, Blainey P C. Optical Pooled Screens in Human Cells. Cell 2019; 179:787-799.e17. https://doi.org/10.1016/j.cell.2019.09.016.
17 Wang C, Lu T, Emanuel G, Babcock H P, Zhuang X. Imaging-based pooled CRISPR screening reveals regulators of IncRNA localization. Proc Natl Acad Sci 2019; 116:10842-51. https://doi.org/10.1073/PNAS.1903808116.
18 Emanuel G, Moffitt J R, Zhuang X. High-throughput, image-based screening of pooled genetic-variant libraries. Nat Methods 2017; 14:1159-62. https://doi.org/10.1038/nmeth.4495.
19 Wang X, Allen W E, Wright M A, Sylwestrak E L, Samusik N, Vesuna S, Evans K, Liu C, Ramakrishnan C, Liu J, Nolan G P, Bava F A, Deisseroth K. Three-dimensional intact-tissue sequencing of single-cell transcriptional states. Science 2018; 361:eaat5691. https://doi.org/10.1126/science.aat5691.
20 A C P, Z D C, P L R, S M M, E M M, C C Y, S M, A S E, A S L, R J, G M C, E S B, J D B, F C. In situ genome sequencing resolves DNA sequence and structure in intact biological samples. Science 2020. https://doi.org/10.1126/SCIENCE.AAY3446.
21 Ke R, Mignardi M, Pacureanu A, Svedlund J, Botling J, Wahlby C, Nilsson M. In situ sequencing for RNA analysis in preserved tissue and cells. Nat Methods 2013; 10:857-60. https://doi.org/10.1038/nmeth.2563.
22 Alon S, Goodwin D, Sinha A, Wassie A, Chen F, Daugharthy E, Bando Y, Kajita A, Xue A, Marrett K, Prior R, Cui Y, Payne A, Yao C C, Suk H J, Wang R, Yu C C, Tillberg P, Reginato P, Pak N, Liu S, Punthambaker S, Iyer E, Kohman R, Miller J, Lein E, Lako A, Cullen N, Rodig S, Helvie K, Abravanel D, Wagle N, Johnson B, Klughammer J, Slyper M, Waldman J, Jane-Valbuena J, Rozenblatt-Rosen O, Regev A, Church G, Marblestone A, Boyden E. Expansion Sequencing: Spatially Precise In Situ Transcriptomics in Intact Biological Systems. BioRxiv 2020:2020.05.13.094268. https://doi.org/10.1101/2020.05.13.094268.
23 Jin X, Demere Z, Nair K, Ali A, Ferraro G B, Natoli T, Deik A, Petronio L, Tang A A, Zhu C, Wang L, Rosenberg D, Mangena V, Roth J, Chung K, Jain R K, Clish C B, Vander Heiden M G, Golub T R. A metastasis map of human cancer cell lines. Nature 2020; 588:331-6. https://doi.org/10.1038/s41586-020-2969-2.

What is claimed is:

1. A method of decoding a nucleic acid barcode in situ in a sample, comprising the following steps:
a) amplifying at least a target region of a segment of the nucleic acid barcode by rolling circle amplification to generate amplified nucleic acids comprising copies of the segment target region, wherein the segment target region comprises one of a plurality of unique primary decoder sequences;
b) contacting the sample with one or more labeled readout probes under conditions that allow hybridization of said labeled readout probes to said amplified nucleic acids, wherein each said labeled readout probe comprises a sequence complementary to a sequence in said amplified nucleic acids;
c) detecting the label(s) of the one or more labeled readout probes; and
d) determining, based on the presence and/or identity of the labeled readout probe, the identity of the nucleic acid barcode.

2. The method of claim 1, wherein each said labeled readout probe comprises a sequence complementary to one of the plurality of unique primary decoder sequences.

3. The method of claim 1 or 2, wherein the nucleic acid barcode comprises a plurality of segments, and each segment comprises a target region comprising one of a unique plurality of unique primary decoder sequences, and wherein
step (a) comprises amplifying at least a target region of each segment of the nucleic acid barcode to generate a set of amplified nucleic acids,
step (b) comprises contacting the sample with one or more labeled readout probes under conditions that allow hybridization of said labeled readout probes to said amplified nucleic acids amplified from a first segment,
step (d) comprises determining, based on the presence and/or identity of the labeled readout probe from each segment, the identity of the nucleic acid barcode, and
wherein the method further comprises the following steps after step (c) and prior to step (d):
e) optionally eliminating signal from the label(s) of the readout probe detectable in step (c); and
f) repeating steps (b), (c), and (e) until the presence and/or identity of the labeled readout probe has been determined for each segment.

4. The method of any of claims 1-3, wherein step (a) comprises the following steps:
a1) contacting the sample with a pair of oligonucleotide probes under conditions that allow hybridization of said oligonucleotide probes to their respective target sequences, said pair of oligonucleotide probes comprising:
(i) a padlock probe comprising at least a region that is complementary to a first part of the segment of the nucleic acid barcode, wherein the padlock probe comprises a 5′ end region and 3′ end region and wherein upon said hybridization the 5′ and 3′ end regions of the padlock probe are brought into juxtaposition for circularization of the padlock probe, and wherein when circularized the padlock probe comprises the reverse complementary sequence of the segment target region; and
(ii) a linear probe comprising a region that is complementary to at least a region of the padlock probe;
a2) circularizing the padlock probe to form a circular padlock probe; and
a3) amplifying the circular padlock probe in situ to generate the amplified nucleic acids comprising copies of the segment target region.

5. The method of claim 4, wherein the linear probe comprises a binding region and an overhang region, wherein the binding region is complementary to a second part of the segment of the nucleic acid barcode and the overhang region is complementary to the at least a region of the padlock probe.

6. The method of claim 5, wherein when the nucleic acid barcode comprises a plurality of segments, the second part of each segment comprises a unique sequence.

7. The method of claim 5, wherein when the nucleic acid barcode comprises a plurality of segments, the second part of each segment comprises the same sequence.

8. The method of claim 7, wherein the linear probe used for each segment comprises the same sequence.

9. The method of any one of claims 5-8, wherein the 5′ and 3′ end regions of the padlock probe are hybridized to the overhang region of the linear probe.

10. The method of any one of claims 4-8, wherein the 5′ and 3′ end regions of the padlock probe are hybridized to the first part of the segment of the nucleic acid barcode.

11. The method of claim 4 or 10, wherein the linear probe does not comprise a binding region that is complementary to any part of the nucleic acid barcode.

12. The method of any one of claims 4, 10, and 11, wherein the segment does not comprise a second part.

13. The method of any one of claims 4-12, wherein upon padlock probe hybridization, the 5′ and 3′ end regions of the padlock probe are immediately next to each other, and wherein the circularizing step is performed by ligating the 5′ and 3′ end regions of the padlock probe with a ligase.

14. The method of any one of claims 4-12, wherein upon padlock probe hybridization, the 5′ and 3′ end regions of the padlock probe have a gap of one or more nucleotides between each other, and wherein the circularizing step is performed by gap filling with a polymerase or a reverse transcriptase, and ligating the 5′ and 3′ end regions of the padlock probe with a ligase.

15. The method of claim 14, wherein when the 5′ and 3′ end regions of the padlock probe are hybridized to the first part of the segment of the nucleic acid barcode, the length of the gap is shorter than the length of the target region.

16. The method of claim 14, wherein when the 5′ and 3′ end regions of the padlock probe are hybridized to the first part of the segment of the nucleic acid barcode, the length of the gap is the same or longer than the length of the target region.

17. The method of any one of claims 4-16, wherein the padlock probe further comprises one of a plurality of unique secondary decoder sequences, wherein each said unique secondary decoder sequence is matched with one of the plurality of unique primary decoder sequences, and wherein the unique secondary decoder sequence is also amplified during rolling circle amplification.

18. The method of claim 17, wherein each said labeled readout probe comprises a sequence complementary to one of the plurality of unique secondary decoder sequences.

19. A method of decoding a nucleic acid barcode in situ in a sample, wherein each nucleic acid barcode comprises a plurality of segments, each segment comprising a target region comprises one of a unique plurality of unique primary decoder sequences, the method comprising the following steps:
a) contacting the sample with a plurality of pairs of oligonucleotide probes under conditions that allow hybridization of said pairs of oligonucleotide probes to their respective target sequences in each segment, each said pair of oligonucleotide probes comprising:
(i) a padlock probe comprising a 5′ end region, a 3′ end region, and a binding region that is complementary to a first part of the segment of the nucleic acid barcode, wherein said first part of the segment comprises a target region of a segment of the nucleic acid barcode and said segment target region comprises one of a plurality of unique primary decoder sequences; and
(ii) a linear probe comprising a binding region and an overhang region, wherein the binding region is complementary to the second part of the segment, wherein the 5′ end and 3′ end regions of the padlock probe are hybridized to the overhang region of the linear probe and wherein upon said hybridization the 5′ and 3′ end regions of the padlock probe are brought into juxtaposition for circularization of the padlock probe;
b) circularizing the padlock probes to form circular padlock probes;
c) amplifying the circular padlock probes in situ to generate a set of amplified nucleic acids comprising copies of the segment target region;
d) contacting the sample with one or more labeled readout probes under conditions that allow hybridization of said labeled readout probes to said amplified nucleic acids amplified from a first segment, wherein each said labeled readout probe comprises a sequence complementary to one of the plurality of the unique primary decoder sequences in the first segment target region;
e) detecting the label(s) of the one or more labeled readout probes;
f) optionally eliminating signal from the label(s) of the readout probe detectable in step (e);
g) repeating steps (d), (e), and (f) until the presence and/or identity of the labeled readout probe has been determined for each segment; and
h) determining, based on the presence and/or identity of the labeled readout probe from each segment, the identity of the nucleic acid barcode.

20. The method of any one of claims 1-19, wherein the barcode comprises 1 to 100 segments.

21. The method of claim 20, wherein the barcode comprises about 10 segments.

22. The method of any one of claims 1-21, wherein the number of unique primary decoder sequences for each segment is about 2 to about 10000.

23. The method of any one of claims 1-22, wherein the number of unique primary decoder sequences for each segment is 3.

24. The method of any one of claims 1-23, wherein the length of each segment is about 15 nucleotides to about 10000 nucleotides.

25. The method of any one of claims 1-24, wherein the length of each segment is about 40 nucleotides.

26. The method of any one of claims 1-25, wherein each segment separated by a spacer.

27. The method of claim 26, wherein the length of the spacer is about 0 nucleotide to about 5000 nucleotides.

28. A method of decoding a nucleic acid barcode in situ in a sample, comprising the following steps:
a) amplifying at least a target region of a nucleic acid barcode by rolling circle amplification to generate amplified nucleic acids comprising copies of the target region, wherein the target region comprises a primary variable sequence;
b) contacting the sample with a plurality of encoding probes under conditions that allow hybridization of said encoding probes to said amplified nucleic acids, wherein at least one of said encoding probe comprises a sequence complementary to a sequence in said amplified nucleic acids, and each said encoding probe comprises one or more of a plurality of unique readout regions;
c) contacting the sample with one or more labeled readout probes under conditions that allow hybridization of said labeled readout probes to said encoding probe(s), wherein each said labeled readout probe comprises a sequence complementary to a unique readout region;
d) detecting the label(s) of the one or more labeled readout probes;
e) optionally eliminating signal from the label(s) of the readout probes detectable in step (d);
f) optionally repeating steps (c), (d) and (e) with one or more additional labeled readout probes, each comprising a sequence complementary to a different unique readout region; and
g) determining, based on the presence of the one or more labeled readout probes, the identity of the nucleic acid barcode.

29. The method of claim 28, wherein the nucleic acid barcode comprises only one target region.

30. The method of claim 28 or 29, wherein at least one of said encoding probes comprises a sequence complementary to the primary variable sequence.

31. The method of any of claims 28-30, wherein each encoding probe comprises two or more of a plurality of unique readout regions.

32. The method of any of claims 28-30, wherein each encoding probe comprises one of a plurality of unique readout regions.

33. The method of any of claims 28-30, wherein the nucleic acid barcode is from a library of nucleic acid barcodes.

34. The method of any of claims 28-33, wherein step (a) comprises the following steps:
a1) contacting the sample with a pair of oligonucleotide probes under conditions that allow hybridization of said oligonucleotide probes to their respective target sequences, said pair of oligonucleotide probes comprising:
(i) a padlock probe comprising at least a region that is complementary to a first part of the nucleic acid barcode, wherein the padlock probe comprises a 5′ end region and 3′ end region and wherein upon said hybridization the 5′ and 3′ end regions of the padlock probe are brought into juxtaposition for circularization of the padlock probe, and wherein when circularized the padlock probe comprises the reverse complementary sequence of the target region; and
(ii) a linear probe comprising a region that is complementary to at least a region of the padlock probe;
a2) circularizing the padlock probe to form a circular padlock probe; and
a3) amplifying the circular padlock probe in situ to generate the amplified nucleic acids comprising copies of the target region.

35. The method of claim 34, wherein the linear probe comprises a binding region and an overhang region, wherein the binding region is complementary to a second part of the nucleic acid barcode and the overhang region is complementary to at least a region of the padlock probe.

36. The method of claim 35, wherein the 5′ and 3′ end regions of the padlock probe are hybridized to the overhang region of the linear probe.

37. The method of claim 34 or 35, wherein the 5′ and 3′ end regions of the padlock probe are hybridized to the first part of the nucleic acid barcode.

38. The method of claim 34 or 37, wherein the linear probe does not comprise a binding region that is complementary to any part of the nucleic acid barcode.

39. The method of any of claims 34-38, wherein when the nucleic acid barcode is from a library of nucleic acid barcodes, the second part of each nucleic acid barcode comprises a unique sequence.

40. The method of any of claims 34-38, wherein when the nucleic acid barcode is from a library of nucleic acid barcodes, the second part of each nucleic acid barcode comprises the same sequence.

41. The method of any one of claims 34, 37, and 38, wherein the nucleic acid barcode does not comprise a second part.

42. The method of any one of claims 34-41, wherein upon padlock probe hybridization, the 5′ and 3′ end regions of the padlock probe are immediately next to each other, and wherein the circularizing step is performed by ligating the 5′ and 3′ end regions of the padlock probe with a ligase.

43. The method of any one of claims 34-41, wherein upon padlock probe hybridization, the 5′ and 3′ end regions of the padlock probe have a gap of one or more nucleotides between each other, and wherein the circularizing step is performed by gap filling with a polymerase or a reverse transcriptase and ligating the 5′ and 3′ end regions of the padlock probe with a ligase.

44. The method of claim 43, wherein when the 5′ and 3′ end regions of the padlock probe are hybridized to the first part of the nucleic acid barcode, the length of the gap is shorter than the length of the target region.

45. The method of claim 43, wherein when the 5′ and 3′ end regions of the padlock probe are hybridized to the first part of the nucleic acid barcode, the length of the gap is the same or longer than the length of the target region.

46. The method of any one of claims 34-45, wherein the padlock probe further comprises a secondary variable sequence, wherein said secondary variable sequence is matched with the primary variable sequence, and wherein the secondary variable sequence is also amplified during rolling circle amplification.

47. The method of claim 46, wherein at least one of said encoding probes comprises a sequence complementary to the secondary variable sequence.

48. A method of decoding a nucleic acid barcode in situ in a sample, wherein the nucleic acid barcode comprises only one target region, said target region comprises a primary variable sequence, said method comprising the following steps:
a) contacting the sample with a pair of oligonucleotide probes under conditions that allow hybridization of said oligonucleotide probes to their respective target sequences, said pair of oligonucleotide probes comprising:
(i) a padlock probe comprising a 5′ end region, a 3′ end region, and a binding region that is complementary to a first part of the nucleic acid barcode, wherein said first part comprises the target region of the nucleic acid barcode; and
(ii) a linear probe comprising a binding region and an overhang region, wherein the binding region is complementary to the second part of the nucleic acid barcode, wherein the 5′ end and 3′ end regions of the padlock probe are hybridized to the overhang region of the linear probe and wherein upon said hybridization the 5′ and 3′ end regions of the padlock probe are brought into juxtaposition for circularization of the padlock probe;
b) circularizing the padlock probes to form circular padlock probes;
c) amplifying the circular padlock probe in situ to generate amplified nucleic acids comprising copies of the target region;
d) contacting the sample with a plurality of encoding probes under conditions that allow hybridization of said encoding probes to said amplified nucleic acids, wherein at least one of said encoding probe comprises a sequence complementary to the primary variable sequence, and each said encoding probe comprises one or more of a plurality of unique readout regions;
e) contacting the sample with one or more labeled readout probes under conditions that allow hybridization of said labeled readout probes to said encoding probe(s), wherein each said labeled readout probe comprises a sequence complementary to a unique readout region;
f) detecting the label(s) of the one or more labeled readout probes;
g) optionally eliminating signal from the label(s) of the readout probe(s) detectable in step (f);
h) optionally repeating steps (e), (f) and (g) with one or more additional labeled readout probes, each comprising a sequence complementary to a different unique readout region; and
i) determining, based on the presence of the one or more labeled readout probes, the identity of the nucleic acid barcode.

49. The method of claim 48, wherein each encoding probe comprises two or more of a plurality of unique readout regions.

50. The method of claim 48, wherein each encoding probe comprises one of a plurality of unique readout regions.

51. The method of any of claims 48-50, wherein the nucleic acid barcode is from a library of nucleic acid barcodes.

52. The method of claim 51, wherein the second part of each nucleic acid barcode comprises a unique sequence.

53. The method of claim 51, wherein the second part of each nucleic acid barcode comprises the same sequence.

54. The method of any one of claims 28-53, wherein the number of unique readout regions is about 2 to 6000.

55. The method of any one of claims 28-54, wherein the length of the variable sequence is about 15 nucleotides to about 300 nucleotides.

56. The method of claim 55, wherein the length of the variable sequence is about 20 nucleotides.

57. The method of any one of claims 4-27 and 34-56, where the linear and/or padlock probes are single-stranded DNA.

58. The method of any one of claims 4-27 and 34-56, where the linear and/or padlock probes are single-stranded LNA or single-stranded DNA with partial LNA modification(s).

59. The method of any one of claims 4-27 and 34-58, where the linear and padlock probes are added simultaneously.

60. The method of any one of claims 4-27 and 34-58, where the linear and padlock probes are added sequentially.

61. The method of any one of claims 1-60, wherein the amplification step is performed with a rolling circle amplification DNA polymerase.

62. The method of claim 61, wherein the rolling circle amplification DNA polymerase is Phi29, Bst, or Vent exo-DNA polymerase.

63. The method of any one of claims 1-60, wherein the amplification step is performed with a rolling circle amplification RNA polymerase.

64. The method of claim 63, wherein the rolling circle amplification RNA polymerase is T7 RNA polymerase.

65. The method of any one of claims 4-27 and 34-64, wherein the circularization step comprises ligation with a ligase.

66. The method of claim 65, wherein the ligase is a DNA ligase.

67. The method of claim 66, wherein DNA ligase is a T4 DNA ligase, T7 DNA ligase, T3 DNA ligase, Taq DNA ligase, Ampligase, or E. coli DNA ligase.

68. The method of claim 65, wherein the ligase is a SplintR ligase.

69. The method of any one of claims 4-27 and 34-68, where the circularization of the padlock probe is performed in situ.

70. The method of any one of claims 4-27 and 34-68, where the circularization of the padlock probe is performed in vitro prior to contacting the sample with the padlock probe.

71. The method of any one of claims 1-70, wherein the readout probes are labeled with fluorescent dyes.

72. The method of claim 71, wherein at least some readout probes are labeled with the same fluorescent dye.

73. The method of claim 71, wherein at least some readout probes are labeled with different fluorescent dyes.

74. The method of any one of claims 3-73, wherein the fluorescence signal is eliminated by photobleaching, chemical bleaching, chemical cleavage, chemical wash, heat denaturation, nuclease treatment, or a combination thereof.

75. The method of any one of claims 3-73, wherein the fluorescence signal is retained.

76. The method of any one of claims 1-75, where the amplified nucleic acids are crosslinked to the sample.

77. The method of claim 76, wherein the crosslinking is performed by aminoallyl-dUTP spike-in during the amplification step, and post-fixation with paraformaldehyde and/or PEGylated bis(sulfosuccinimidyl)suberate (BS(PEG)5 or BS(PEG)9).

78. The method of any of claims 1-77, wherein the nucleic acid barcode is comprised in a DNA, RNA, locked nucleic acid (LNA), DNA with partial LNA modification(s), or peptide nucleic acid (PNA) molecule.

79. The method of claim 78, wherein the nucleic acid molecule is double-stranded.

80. The method of claim 78, wherein the nucleic acid molecule is single-stranded.

81. The method of any of claims 1-80, where the nucleic acid barcode is delivered into cells.

82. The method of any of claims 1-80, where the barcode is not delivered into cells but is decoded on the surface of cells or independent of cells.

83. The method of any of claims 1-82, where the barcode is decoded at a molecular level, cellular level, or multi-cellular level.

84. A method of performing an in situ genetic screen, comprising:
pairing a genetic screen technique with nucleic acid barcodes;
performing the genetic screen technique; and
decoding the nucleic acid barcodes with a decoding method of any of claims 1-83.

85. The method of claim 84, wherein the genetic screen technique is a pooled genetic screen technique.

86. The method of claim 84 or 85, wherein the genetic screen technique is a CRISPR screen technique.

87. The method of claim 86, wherein the CRISPR screen is a CRISPR knockout screen, a CRISPR interference (CRISPRi) screen, a CRISPR activation (CRISPRa) screen, a CRISPR screen of cis-regulatory elements, a CRISPR screen of protein domain functions, or a CRISPR double-perturbation screen.

88. The method of claim 84 or 85, wherein the genetic screen technique is an RNA interference (RNAi) screen technique.

89. The method of claim 84 or 85, wherein the genetic screen technique is a massively parallel reporter assay screen.

90. The method of any of claims 84-89, wherein the step of pairing a genetic screen technique with nucleic acid barcodes further comprises pairing at least one nucleic acid barcode with at least one nucleic acid genetic perturbation sequence.

91. The method of claim 90, wherein each barcode pairs with a unique genetic perturbation sequence.

92. The method of any of claims 90-91, wherein each barcode and genetic perturbation sequence pairing are located on one polynucleotide sequence.

93. The method of any of claims 90-91, wherein the nucleic acid barcode is attached to the genetic perturbation sequence or is the genetic perturbation sequence.

94. The method of any of claims 90-91, wherein each barcode and genetic perturbation sequence pairing are located on multiple polynucleotide sequences.

95. The method of any of claims 90-94, wherein the step of pairing a genetic screen technique with nucleic acid barcodes comprises pairing at least one nucleic acid barcode with a combination of at least two nucleic acid genetic perturbation sequences.

96. The method of any of claims 90-95, wherein the genetic perturbation sequence is a guide RNA (gRNA).

97. The method of any of claims 90-96, wherein each genetic perturbation sequence is a unique gRNA.

98. The method of any of claims 90-97, wherein the nucleic acid barcodes and nucleic acid genetic perturbation sequences are delivered into cells.

99. The method of any of claims 90-98, wherein the nucleic acid barcodes and nucleic acid genetic perturbation sequences are delivered into cells by viral transduction, transfection, electroporation, or microinjection.

100. The method of any of claims 90-99, wherein the nucleic acid barcodes and nucleic acid genetic perturbation sequences are delivered into cells by viral transduction.

101. The method of claim 100, wherein viral transduction is performed by a lentivirus or an adeno-associated virus (AAV).

102. The method of any of claims 90-101, wherein the method further comprises analyzing the results of the genetic screen technique to determine a phenotypic perturbation.

103. The method of claim 102, wherein the phenotypic perturbation is a perturbation of cell size, cell shape, cellular interactions, cellular abundance/density of biomolecules, sub-cellular abundance/density of biomolecules, sub-cellular morphologies, sub-cellular distribution, and/or sub-cellular organization.

104. The method of claim 102 or 103, wherein the phenotypic perturbation is a perturbation of genome architecture.

105. The method of claim 104, wherein the phenotypic perturbation is a perturbation of three-dimensional chromatin organization.

106. The method of any of claims 102-105, wherein the analysis of the results of the genetic screen technique is performed by an imaging technique.

107. The method of claim 106, wherein the imaging technique is in situ hybridization.

108. The method of claim 106 or 107, wherein the imaging technique is fluorescence in situ hybridization.

109. The method of any of claims 106-108, wherein the imaging technique is multiplexed DNA or RNA fluorescence in situ hybridization.

110. The method of claim 106, wherein the imaging technique is imaging of lipid, sugar, metabolite, DNA, RNA, protein and/or DNA/RNA/protein modifications.

111. The method of any of claims 102-110, wherein the method further comprises the step of matching the decoded nucleic acid barcodes with the determined phenotypic perturbation.

112. The method of claim 111, wherein the matching of the decoded barcode with the phenotypic perturbation allows for the determination of which genetic perturbation sequence matches which phenotypic perturbation.

113. The method of any of claims 102-112, wherein the step of analyzing the results of the genetic screen technique to determine a phenotypic perturbation can be performed prior to, during, or after the decoding step.

114. A method of performing an in situ genetic screen, comprising the following steps:
creating at least one unique pairing of at least one nucleic acid barcode with at least one nucleic acid genetic perturbation sequence;
introducing at least one unique pairing of the at least one barcode and the at least one perturbation sequence to a cell;
incubating the cell under conditions that allow the at least one perturbation sequence to cause the cell to display at least one phenotypic perturbation;
analyzing the cell by an imaging technique to determine the at least one phenotypic perturbation;
decoding the at least one nucleic acid barcode with a decoding method of any of claims 1-83; and
determining the at least one genetic perturbation sequence that causes the cell to display the at least one phenotypic perturbation.

115. A method of determining cellular positions in a single-cell sequencing, comprising the following steps:
introducing at least one nucleic acid barcode to at least one cell;
imaging the at least one cell to determine cellular position;
decoding the nucleic acid barcodes with a decoding method of any of claims 1-83;
dissociating the at least one cell from its substrate;
performing single-cell sequencing on the at least one cell to determine at least the sequence of the nucleic acid barcode associated with the at least one cell; and
mapping the at least one cell to the cellular position.

116. The method of claim 115, wherein the at least one nucleic acid barcode is comprised in a DNA, RNA, locked nucleic acid (LNA), DNA with partial LNA modification(s), or peptide nucleic acid (PNA) molecule.

117. The method of claim 115 or 116, wherein the at least one nucleic acid barcode is delivered by a viral vector.

118. The method of claim 117, wherein the viral vector is a lentivirus or adeno-associated virus (AAV).

119. The method of any of claims 115-118, wherein the method comprises introducing a plurality of nucleic acid barcodes to a plurality of cells, and wherein each nucleic acid barcode is only present in one cell.

120. The method of claim 119, wherein each nucleic acid barcode is a unique nucleic acid barcode.

121. The method of any of claims 115-120, wherein the at least one cell is present in at least one tissue.

122. The method of any of claims 115-121, wherein the step of performing single-cell sequencing on the at least one cell further determines additional genomic information about the at least one cell.

123. The method of any of claims 115-122, wherein the step of performing single-cell sequencing on the at least one cell further determines the gene expression of the at least one cell.

124. The method of any of claims 115-123, wherein the step of performing single-cell sequencing on the at least one cell further determines epigenetic/epigenomic information about the at least one cell.

125. The method of any of claims 115-124, wherein the step of mapping the at least one cell to the cellular position provides spatial-omic information about the at least one cell.


SEQUENCE LISTING (1 sequence)

SEQ ID NO: 1 - DNA, 186 bp, Artificial Sequence
  Description of Artificial Sequence Synthetic polynucleotide
gtggaaaggacgaaacaccgaaaaaacgaacggattaacagttttagagctagaaatagcaagttaaaataaggctagtccgttatcaacttgaaaaagtggcaccgagtcggtgctttttttccgtacggcgcacttagcttaatgggaaggtgaaagtgtaagcttggcgtaactagatcttga